(19) United States
(12) Patent Application Publication
(10) Pub. No.: US 2021/0393701 A1
(43) Pub. Date: Dec. 23, 2021

(54) REGENERATIVE ABSCOPAL EFFECTS

(71) Applicant: Figene, LLC, Houston, TX (US)

(72) Inventors: Pete O'Heeron, Houston, TX (US); Thomas Ichim, San Diego, CA (US)

(21) Appl. No.: 17/309,207

(22) PCT Filed: Nov. 8, 2019
(86) PCT No.: PCT/US2019/060397

§ 371 Date: May 6, 2021

Related U.S. Application Data
(60) Provisional application No. 62/757,764, filed on Nov. 9, 2018.

(51) Int. Cl. A61K 35/33; A61K 38/18; A61K 35/16; A61K 35/19; A61K 35/545; A61K 35/28; A61K 31/7088; A61K 45/06; A61P 19/00; A61P 19/02; A61K 9/00

(52) U.S. Cl. CPC A61K 35/33; A61K 38/18; A61K 35/16; A61K 35/19; A61K 35/545; A61K 9/0019; A61K 31/7088; A61K 45/06; A61P 19/00; A61P 19/02; A61K 9/0085; A61K 35/28

(57) ABSTRACT

Embodiments of the disclosure encompass methods and compositions using fibroblasts for stimulating regeneration in a first tissue site in an individual, comprising the step of administering at least one regenerative composition to a second tissue site, wherein the second tissue site comprises the same tissue type as the first tissue site in the individual. The first and second sites are at different locations in the individual, in particular embodiments. Particular embodiments comprise administering one or more compositions to an individual at a different anatomical site than the site that is in need, such as a joint.

This application claims priority to U.S. Provisional Patent Application Ser. No. 62/757,764, filed Nov. 9, 2018, which is incorporated by reference herein in its entirety.

### TECHNICAL FIELD

Embodiments of the disclosure include at least the fields of cell biology, molecular biology, cell therapy, physiology, and medicine.

### BACKGROUND

Stem cell therapy has substantially advanced in the last two decades. Original uses of stem cells involved the reconstitution of recipient hematopoiesis subsequent to myeloablative treatment in the area of hematological disorders. Subsequent to the initial clinical successes of bone marrow transplantation, much work has been performed demonstrating that bone marrow cells are capable of exerting therapeutic effects in non-hematological areas such as liver failure, heart failure, and limb ischemia.

Localized administration of stem cells has been reported to induce regenerative effects at the site of administration such as joints, muscle, and other tissues. Unfortunately, it is difficult in certain conditions to locally administer stem cells into all of the tissue(s) in need of regeneration. For example, in Duchenne Muscular Dystrophy, it is known that stem cells induce a localized re-expression of dystrophin, however it is difficult to inject stem cells into every muscle of the body.

The present disclosure provides solutions to long-felt needs in the areas of cell therapy using efficient and novel methods.

### BRIEF SUMMARY

The present disclosure is directed to systems, methods, and compositions for regenerating one or more tissues in an individual at one or more sites in need of cell and/or tissue regeneration. Particular embodiments comprise administering one or more compositions to an individual at a different anatomical site than the site that is in need of regeneration. The composition may induce regeneration at the site in need when the administration site comprises the same tissue type as the type of tissue at the site in need of regeneration. As an example, a site in need of regeneration may be a joint, such as the left knee for example, wherein the site of administration is to the right knee. Although, in the provided example the right knee may or may not be in need of regeneration, the composition may, in a way not bound by theory, be characteristically able to induce regeneration in the left knee. Particular embodiments may comprise administration in any tissue, such as muscle tissue, connective tissue, joint tissue, epithelial tissue, endothelial tissue, nervous tissue, fat tissue, skin tissue, lung tissue, liver tissue, bladder tissue, kidney tissue, heart tissue, stomach tissue, intestinal tissue, spinal tissue, eye tissue, fibrous tissue, omentum, or bone marrow, for example, and the tissue may be made of any one or more cell types. Administration of one or more regenerative compositions into any such tissue may then provide and/or induce regeneration in tissue of the same type, but not at the administration site. The disclosure specifically encompasses regeneration at a site that is different than the site of administration.

In certain embodiments, the administration comprises systemic or local delivery of the composition, wherein the administration may be to a specific site and may or may not be by injection. The administration may be a single administration or multiple administrations including 2, 3, 4, 5, 6, 7, 8, 9, 10 or more administrations, including over a specific period of time, in some cases.

In certain embodiments, the site in need of regeneration has partial or full loss of functionality. Partial loss of functionality comprises the tissue not having full function as compared to a healthy tissue of the same type, such as partial loss of glomerular filtration in the kidney for example. Full loss of functionality comprises complete loss of the normal function for the tissue type, such as complete kidney failure for example. Loss of functionality may comprise cell death in the tissue, tissue necrosis, atrophy, fibrosis, inflammation, fat deposition, generation of degenerative molecules, loss of elasticity, neurodegeneration, autoimmunity, complement activation, cartilage loss, ligament tear(s), muscle tear(s), loss of connective tissue, neoplasm(s), for example.

In certain embodiments the composition administered comprises at least one cell, growth factor, blood product, exosome, miRNA, epigenetic-acting composition, or a combination thereof. The cell may be of any type or origin including a fibroblast or stem cell or a mixture thereof. The stem cell could be of any lineage and may be a mesenchymal stem cell, pluripotent stem cell, hematopoietic stem cell, inducible pluripotent stem cell, parthenogenic derived stem cell, or mesenchymal stem cell derived from pluripotent sources, or any other stem cell. The cell may express and/or lack expression of certain cell markers, for example to aid in the identification of the cell being used, as an example, or to produce a certain activity of the cell. The cell could be harvested from any tissue, such as bone marrow, peripheral blood, adipose tissue, mobilized peripheral blood, umbilical cord blood, Wharton's jelly, umbilical cord tissue, skeletal muscle tissue, subepithelial umbilical cord, endometrial tissue, menstrual blood, fallopian tube tissue, foreskin, placenta, ear lobe, omentum, or a combination thereof, for example. The cell could be from an allogenic or autologous or xenogeneic source. The epigenetic-acting composition may be a histone deacetylase inhibitor or may be a histone methyltransferase inhibitor or a combination thereof.

Embodiments of the disclosure include methods of stimulating regeneration in a first tissue site (and the first tissue may or may not be degenerated) in an individual, comprising the step of administering at least one regenerative composition comprising fibroblasts and/or dedifferentiated fibroblast cells and optionally stem cells to a second tissue site, wherein the second tissue site comprises the same tissue type as the first tissue site in the individual. The first and second tissue sites are at different locations of the body of the individual. Administering the composition may comprise systemic injection, local injection, systemic delivery, and/or local delivery. Administering the composition may comprise at least one administration or 2, 3, 4, 5, 6, 7, 8, 9, 10 or more administrations. In specific embodiments, the first tissue has partial or full loss of functionality, such as loss of functionality comprising cell death in the tissue, tissue necrosis, atrophy, fibrosis, inflammation, fat deposition, generation of degenerative molecules, loss of elasticity, neurodegeneration, autoimmunity, complement activation, cartilage loss, ligament tear(s), muscle tear(s), loss of connective tissue, neoplasm(s), or a combination thereof. The tissue may be of any kind, including tissue that is comprised of muscle tissue, connective tissue, epithelial tissue, endothelial tissue, nervous tissue, fat tissue, skin tissue, lung tissue, liver tissue, bladder tissue, kidney tissue, heart tissue, stomach tissue, intestinal tissue, spinal tissue, eye tissue, fibrous tissue, omentum, lymphatic tissue, bone marrow, or a combination thereof. In specific cases, the tissue is comprised of one or more cells selected from the group consisting of endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, interstitial kidney cells, and a combination thereof.

Any regenerative composition may comprise at least one growth factor, such as a growth factor selected from a group consisting of AM, Ang, BMP, BDNF, EGF, Epo, FGF, GNDF, G-CSF, GM-CSF, GDF-9, HGF, HDGF, IGF, migration-stimulating factor, GDF-8, GDF-11, GDF-15, MGF, NGF, P1GF, PDGF, Tpo, TGF-alpha, TGF-beta, TNF-alpha, VEGF, a Wnt protein, an interleukin, a soluble receptor for IL-1alpha, IL-1beta, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12, 35 kDa alpha subunit, IL-12, 40 kDa beta subunit, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-24, IL-25, IL-26, IL-27B, IL-27-p28, IL-28A, IL-28B, IL-29, IL-30, IL-31, IL-32, IL-33, IL-34, IL-35, IL-36alpha, IL-36beta, IL-36gamma, an interferon (IFN), a soluble receptor for IFN-alpha, IFN-beta, IFN-gamma, IFN-lamdal, IFN-lamda2, IFN-lamda3, IFN-K, IFN-epsilon, IFN-kappa, IFN-tau, IFN-delta, IFN-zeta, IFN-omega, IFN-v, insulin, proinsulin, a receptor for insulin, leptin (LEP), and a combination thereof.

In particular embodiments, a regenerative composition comprises platelet rich plasma that may comprise platelet lysate. The platelet rich plasma may be derived from peripheral blood, cord blood, or a mixture thereof. In specific embodiments, the regenerative composition comprises one or more exosome(s) derived from at least one regenerative cell, such as a stem cell and/or a fibroblast. The fibroblast may be derived from tissue sources selected from the group consisting of foreskin, adipose tissue, placenta, ear lobe, omentum, wharton's jelly, and a combination thereof.

When the regenerative composition comprises one or more exosomes, the exosomes may be a size of between about 2 nm and 200 nm, including a size between about 30 and 150 nm. The exosomes may comprise at least one lipid selected from the group consisting of phospholipids, phosphatidyl serine, phosphatidyl inositol, phosphatidyl choline, sphingomyelin, ceramides, glycolipid, cerebroside, steroids, cholesterol, and a combination thereof. The exosomes may comprise at least one lipid raft. In specific embodiments, the exosomes comprise one or more antigenic markers on a surface of the exosomes, and the marker(s) may be selected from the group consisting of CD9, CD63, CD81, ANXA2, ENO1, HSP9OAA1, EEF1A1, YWHAE, SDCBP, PDCD6IP, ALB, YWHAZ, EEF2, ACTG1, LDHA, HSP90AB1, ALDOA, MSN, ANXAS, PGK1, CFL1, and a combination thereof.

When the regenerative composition comprises one or more fibroblasts, they may be derived from any suitable source, such as derived from tissue sources selected from the group consisting of foreskin, adipose tissue, placenta, ear lobe, adipose tissue, omentum, wharton's jelly, and a combination thereof. The fibroblasts may express at least one marker selected from the group consisting of NANOG, OCT-4, SSEA-4, stem cell factor receptor, and a combination thereof. Any regenerative composition that comprises fibroblasts may also comprise stem cells.

When the regenerative composition comprises one or more stem cells, the stem cells may be of any kind. Examples include pluripotent stem cells, such as pluripotent stem cells selected from the group consisting of hematopoietic stem cells, embryonic stem cells, parthenogenic derived stem cells, inducible pluripotent stem cells, somatic cell nuclear transfer derived stem cells, cytoplasmic transfer derived stem cells, stimulus-triggered acquisition of pluripotency, and a combination thereof. Hematopoietic stem cells that may be used may be capable of multi-lineage reconstitution in an immunodeficient host. The hematopoietic stem cells may express at least one of the proteins selected from the group consisting of c-kit, Sca-1, CD34, CD133, and a combination thereof. In some cases, the hematopoietic stem cells lack expression of one or more lineage markers, such as CD38, CD14, CD16, CD56, or a combination thereof. The hematopoietic stem cell may be positive for expression of c-kit, positive for expression of Sca-1, and/or substantially lacks expression of lineage markers. Hematopoietic stem cells may be derived from sources selected from the group consisting of peripheral blood, mobilized peripheral blood, bone marrow, cord blood, adipose stromal vascular fraction, derived from progenitor cells, and a combination thereof. In specific embodiments, the hematopoietic progenitor cell is a pluripotent stem cell. Stem cells when utilized may comprise mesenchymal stem cells, including mesenchymal stem cells that are plastic adherent. The mesenchymal stem cells may express a marker selected from the group consisting of CD73, CD90, CD105, and a combination thereof. The mesenchymal stem cells may lack expression of a marker selected from the group consisting of CD14, CD45, CD34, and a combination thereof. In specific embodiments, the mesenchymal stem cells are derived from tissues selected from the group consisting of bone marrow, peripheral blood, adipose tissue, mobilized peripheral blood, umbilical cord blood, Wharton's jelly, umbilical cord tissue, skeletal muscle tissue, subepithelial umbilical cord, endometrial tissue, menstrual blood, fallopian tube tissue, and a combination thereof. The mesenchymal stem cells may be derived from umbilical cord tissue express markers selected from the group consisting of oxidized low density lipoprotein receptor 1, chemokine receptor ligand 3, granulocyte chemotactic protein, and a combination thereof. In specific cases, the mesenchymal stem cells from umbilical cord tissue do not express markers selected from the group consisting of CD117, CD31, CD34, CD45, and a combination thereof. The mesenchymal stem cells from umbilical cord tissue may express, relative to a human fibroblast, increased levels of interleukin 8 and/or reticulon 1, in some cases. In specific embodiments, the mesenchymal stem cells from umbilical cord tissue express markers selected from the group consisting of CD10, CD13, CD44, CD73, CD90, and a combination thereof. Umbilical cord tissue-derived cell secretes factors may be selected from the group consisting of MCP-1, MIP1beta, IL-6, IL-8, GCP-2, HGF, KGF, FGF, HB-EGF, BDNF, TPO, RANTES, TIMP1, and a combination thereof. The umbilical cord tissue-derived cells may express markers selected from the group consisting of TRA1-60, TRA1-81, SSEA3, SSEA4, NANOG, and a combination thereof. The umbilical cord tissue-derived mesenchymal stem cells may be isolated umbilical cord tissue cells isolated from umbilical cord tissue substantially free of blood that is capable of self-renewal and expansion in culture. The umbilical cord tissue-derived cells may be positive for alkaline phosphatase staining. In specific embodiments, the cord tissue-derived mesenchymal stem cells can undergo at least 20 doublings in culture. The cord tissue-derived mesenchymal stem cells may maintain a normal karyotype upon passaging. In some cases, the umbilical cord tissue-derived mesenchymal stem cells express a marker selected from the group consisting of CD10, CD13, CD44, CD73, CD90, PDGFr-alpha, PD-L2, HLA-A,B,C, and a combination thereof. The cord tissue-derived mesenchymal stem cells may not express one or more markers selected from the group consisting of CD31, CD34, CD45, CD80, CD86, CD117, CD141, CD178, B7-H2, HLA-G, HLA-DR,DP,DQ, and a combination thereof. Bone marrow-derived mesenchymal stem cells express markers may be selected from the group consisting of LFA-3, ICAM-1, PECAM-1, P-selectin, L-selectin, CD49b/CD29, CD49c/CD29, CD49d/CD29, CD29, CD18, CD61, 6-19, thrombomodulin, telomerase, CD10, CD13, CD34, CD56, CD117, integrin beta, and a combination thereof. Bone marrow mesenchymal stem cells may not express CD10. B marrow mesenchymal stem cells may not express at least one of CD2, CD5, CD14, CD19, CD33, CD45, and/or DRII. Bone marrow mesenchymal stem cells may express at least one of CD13,CD34, CD56, CD90, CD117 and/or nestin. In specific embodiments, the bone marrow-derived mesenchymal stem cells comprise mesenchymal stem cell progenitor cells. The mesenchymal progenitor cells comprise a population of bone marrow mesenchymal stem cells enriched for cells expressing STRO-1. In specific embodiments, mesenchymal progenitor cells express both STRO-1 and VCAM-1. STRO-1 expressing cells may be negative for at least one marker selected from the group consisting of CBFA-1, collagen type II, PPAR.gamma2, osteopontin, osteocalcin, parathyroid hormone receptor, leptin, H-ALBP, aggrecan, Ki67, glycophorin A, and a combination thereof. In specific embodiments, bone marrow mesenchymal stem cells lack expression of at least one of CD14, CD34, and/or CD45. STRO-1 expressing cells may be positive for a marker selected from the group consisting of VCAM-1, TKY-1, CD146, STRO-2, and the combination thereof. In specific embodiments, skeletal muscle stem cells express markers selected from the group consisting of CD13, CD34, CD56, CD117, and a combination thereof. Skeletal muscle mesenchymal stem cells may not express CD10. In specific embodiments, skeletal muscle mesenchymal stem cells do not express at least one of CD2, CD5, CD14, CD19, CD33, CD45, and/or DRII. Subepithelial umbilical cord-derived mesenchymal stem cells may be utilized and may possess markers selected from the group consisting of CD29, CD73, CD90, CD166, SSEA4, CD9, CD44, CD146, CD105, and a combination thereof. Subepithelial umbilical cord derived mesenchymal stem cells may not express markers selected from the group consisting of CD45, CD34, CD14, CD79, CD106,CD86, CD80, CD19, CD117, Stro-1, HLA-DR, and a combination thereof. In specific embodiments, subepithelial umbilical cord derived mesenchymal stem cells express at least one of CD29, CD73, CD90, CD166, SSEA4, CD9, CD44, CD146, and/or CD105. In specific cases, subepithelial umbilical cord derived mesenchymal stem cells do not express at least one of CD45, CD34, CD14, CD79, CD106, CD86, CD80, CD19, CD117, Stro-1, and/or HLA-DR. Subepithelial umbilical cord derived mesenchymal stem cells may be positive for SOX2 and/or OCT4.

In particular embodiments, regenerative composition comprises one or more fibroblast derived apoptotic vesicles. The regenerative composition may comprise fibroblast-derived miRNAs; fibroblast derived miRNAs may comprise in exosomes; fibroblast derived miRNAs are comprised in apoptotic bodies; and/or fibroblast derived miRNAs are circulating in plasma. In specific embodiments, there is enhancement of one or more distant regenerative effects. In specific embodiments, this is accomplished by systemic administration (for example, at the site of administration of the regenerative composition or at a different site) of one or more epigenetic-acting compositions, such as one or more histone deacetylase inhibitors; one or more DNA methyltransferase inhibitors.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

In reviewing the detailed disclosure which follows, and the specification more generally, it should be borne in mind that all patents, patent applications, patent publications, technical publications, scientific publications, and other references referenced herein are hereby incorporated by reference in this application, in their entirety to the extent not inconsistent with the teachings herein. It is important to an understanding of the present disclosure to note that all technical and scientific terms used herein, unless defined herein, are intended to have the same meaning as commonly understood by one of ordinary skill in the art. The techniques employed herein are also those that are known to one of ordinary skill in the art, unless stated otherwise. It is also to be understood that this disclosure is not limited to the specific embodiments and methods described below, as specific components and/or conditions may, of course, vary. Furthermore, the terminology used herein is used only for the purpose of describing particular embodiments of the present disclosure and is not intended to be limiting in any way.

### I. Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, the word “comprising” is synonymous with “including,” “having,” “containing,” or “characterized by.” These terms are inclusive and open-ended and do not exclude additional, unrecited elements or method steps. In accordance with the present disclosure, the phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole. In accordance with the present disclosure, the phrase “consisting essentially of” limits the scope of a claim to the specified materials or steps, plus those that do not materially affect the basic and novel characteristic(s) of the claimed subject matter.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “cell culture” and “culturing of cells” refer to the maintenance and propagation of cells comprising human, human-derived, and animal cells in vitro, in particular embodiments. The cells may be fibroblasts, stem cells, or a mixture thereof.

As used herein, the term “cell culture medium” refers to the maintenance of cells in culture in vitro. For some cell types, the medium may also be sufficient to support the proliferation of the cells in culture. A medium according to the present disclosure may comprise one or more nutrients such as energy sources, amino acids and/or inorganic ions, for example. Additionally, it may comprise a dye (such as phenol red), sodium pyruvate, several vitamins, free fatty acids, antibiotics, anti-oxidants and/or trace elements. For culturing fibroblasts or mesenchymal stem cells that are dedifferentiated into stem cells, or stem cell-like cells according to the present disclosure, any standard medium such as Iscove's Modified Dulbecco's Media (IMDM), alpha-MEM, Dulbecco's Modified Eagle Media (DMEM), RPMI Media and McCoy's Medium, for example, may be suitable before reprogramming. Once the cells have been reprogrammed, they may, in particular embodiments, be cultured, for example in embryonic stem cell medium.

As used herein, the term “derived from” refers to obtaining a composition from a specific source in a manner that retains, at least in part, desirable properties from the source. Examples include fibroblasts isolated and obtained from any suitable, such as adipose tissue as an example, wherein the isolated fibroblasts maintain, at least in part, characteristics of fibroblasts that are found in adipose tissue. Isolated fibroblasts in the provided example are described as fibroblasts derived from adipose tissue.

As used herein, the term “dedifferentiate” or “dedifferentiation” refers to the process by which lineage-committed cells, myoblasts or osteoblasts for example, reverse their lineage commitment and become precursor or progenitor cells, multipotent or pluripotent stem cells for example. Dedifferentiated cells may, for instance, be identified by loss of patterns of gene expression and cell surface protein expression associated with the lineage committed cells. In some aspects, a dedifferentiated cell acquires one or more characteristics previously possessed by that cell type at an earlier developmental time point. An example of dedifferentiation is the temporal loss of epithelial cell characteristics during wounding and healing. Dedifferentiation can occur in degrees. In the aforementioned example of wound healing, dedifferentiation progresses only slightly before the cells redifferentiate to recognizable epithelia. A cell that has greatly dedifferentiated, for example, is one that resembles a stem cell. Dedifferentiated cells may remain dedifferentiated and proliferate as a dedifferentiated cell; redifferentiate along the same developmental pathway from which the cell had previously dedifferentiated; or redifferentiate along a developmental pathway distinct from which the cell had previously dedifferentiated. Within the context of the present disclosure, a dedifferentiated mesenchymal stem cell possesses enhanced plasticity and ability to differentiate, or redifferentiate into other cells. The dedifferentiated state of the treated cell, which in the current disclosure may be a mesenchymal stem cell, can be verified by increased expression of one or more genes selected from the group consisting of alkaline phosphatase (ALP), OCT4, SOX2, human telomerase reverse transcriptase (hERT), SSEA-4, and a combination thereof. The cells may be verified by positive alkaline phosphatase staining, as an example. That is, the somatic cells introduced with the reprogramming gene (such as OCT4, Nanog, or Sox-2) are treated with a dedifferentiation agent such as valproic acid, and then an initial process in which a colony is generated in the dedifferentiation process is observed through alkaline phosphatase staining (AP staining), and furthermore, expression of Oct4 is verified by immunofluorescence (IF) using an Oct4 antibody. In some cases, fibroblasts are dedifferentiated into another type of cell prior to its use in methods of the disclosure.

As used herein, the term “differentiate” or “differentiation” refers to the process by which precursor or progenitor cells, for example stem cells, differentiate into specific cell types, for example osteoblasts, or cells of specific tissue types, such as skeletal muscle, vascular smooth muscle, pericyte, or vascular endothelium, for example, or cells of specific phenotypes such as osteocytic differentiation, adipogenic differentiation, or chondrogenic differentiation, for example. Differentiated cells may be identified by their patterns of gene expression and cell surface protein expression.

As used herein, the term “express” when referring to a cell expressing a particular protein, marker, and/or gene means the cells contains or comprises the particular protein, marker, and/or the transcribed RNA of the particular gene. The cell may have transcribed or be actively transcribing the particular gene. The cell may have translated or be actively translating the mRNA coding for the particular protein and/or marker. In particular embodiments, the cell can be determined to express or be expressing a particular protein, marker, and/or gene if there are detectable amounts of the particular protein, marker, and/or gene. Any method known in the art to detect proteins, makers, and/or genes may be used including Western blots, flow cytometry, mass spectrometry, PCR, qPCR, RT-qPCR, Southern blotting, ELISA, and/or designed kits. Conversely, when a cell, or cells, is/are said to lack expression or do(es) not express a particular protein, marker, and/or gene, there may not be detectable amounts of that particular protein, marker, and/or gene.

As used herein, the term “homologous tissues” refers to two or more tissues or tissue sites that are of the same type and have the same function. Homologous tissues in an individual include, for example, a left knee and a right knee, a left kidney and a right kidney, a left lung and a right lung, a vertebra and any other vertebrae, an intervertebral disc and all other intervertebral discs, a left eye and a right eye, a left calf and a right calf, or any other tissue that has the same tissue type at a different anatomical location in the individual. Homologous tissues also encompass muscle types, such as skeletal muscle tissues, cardiac muscle tissues, and smooth muscle tissues. For example, skeletal muscle tissue in an arm is homologous to skeletal muscle tissue in a thigh. For purposes of convenience, homologous tissues also refer to cell types that are homologous, such as hematopoietic cells of various lineages.

As used herein, the term “reprogramming” or “remodeling” refers in general to altering epigenetic markers of a cell such as DNA methylation, histone methylation, histone acetylation, for example, or activating genes by inducing transcription factor signal systems, such as for Oct4, for example. In particular, a reprogramming embodiment of the present disclosure provides at least one dedifferentiated and/or rejuvenated cell. Particular embodiments provide administration of a cell that has the characteristics of a multipotent, in particular pluripotent, stem cell. Thus, in case the cell to be reprogrammed is a cell that already has a multipotent or pluripotent character, the present disclosure is able to maintain these cells by the reprogramming of the present disclosure in their multi- or pluripotent state for a prolonged period of time. In specific embodiments, wherein the cells to be administered possess multipotent or pluripotent characteristics, the cells may be passaged for a duration before administration. The cells may be passaged 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more times before administration. The cells may maintain their normal karyotype throughout passaging. In case the cells to be reprogrammed are in an aged or differentiated state, the cell may undergo dedifferentiation into a multipotent or pluripotent stem cell. In particular embodiments, multipotent cells may be reprogrammed to become pluripotent cells.

As used herein, the word “stem cell” refers to any self-renewing pluripotent cell or multipotent cell or progenitor cell or precursor cell that is capable of differentiating into one or multiple cell types. Stem cells may differentiate into one, or more than one, cell type and may have an unlimited growth potential. Stem cells may include those that are capable of differentiating into cells of an osteoblast lineage or a mesenchymal cell lineage (e.g. bone, cartilage, adipose, muscle, stroma, including hematopoietic supportive stroma, and tendon).

As used herein, the term “transfection” refers to a method of gene delivery that introduces a foreign nucleotide sequences into a cell preferably by a viral or non-viral method. In embodiments of to the present disclosure, foreign DNA/RNA/proteins may be introduced to a cell by transient transfection of an expression vector encoding a polypeptide of interest, whereby the foreign DNA/RNA/proteins is introduced but eliminated over time by the cell and during mitosis. As used herein, the term “transient transfection” refers to a method where the introduced expression vectors and the polypeptide encoded by the vector are not permanently integrated into the genome of the host cell, or anywhere in the cell, and therefore may be eliminated from the host cell or its progeny over time. Proteins, polypeptides, or other compounds may also be delivered into a cell using transfection methods. The cells of the disclosure may be transfected, in certain embodiments,

### II. General Methods

Disclosed are means, methods and compositions of matter useful for eliciting a systemic (or local), tissue-specific, regenerative effect subsequent to administration of at least one regenerative composition into one or more tissues in order to induce regeneration of one or more homologous tissues at different anatomical positions. The administration of at least one regeneration composition into the tissue that is stimulated to regenerate may induce a whole body effect resulting in regeneration of non-administered tissues possessing homology to the tissue administered with the regenerative composition. In some embodiments, the disclosure encompasses regeneration of a homologous tissue such as a proximal or distal vertebral disc subsequent to administration of a regenerative composition to a disc. In specific embodiments of the disclosure, a bilateral tissue such as a kidney, a joint, or a series of joints are induced to regenerate as a result of administration of a regenerative means to a kidney or joint, respectively, on a different part of the body. In particular embodiments, the regenerative process occurs at a distant location to the site of administration. Such embodiments may be amplified by administration of one or more agents capable of inducing stem cell mobilization.

The disclosure provides means of inducing regeneration at a site distal to an area of the body administered with one or more regenerative compositions. In specific embodiments of the disclosure, one or more fibroblasts, or any cell(s) of the present disclosure, may be administered as at least part of a regenerative composition in one area of the body, with the result of inducing regeneration in at least one distal area of the body. Regenerative compositions may comprise fibroblasts and/or dedifferentiated fibroblast cells and optionally stem cells.

Specifically, the administration of a regenerative composition is performed in an area of the body homologous to the area in which regeneration is desired. In particular embodiments, the tissue that receives the administered regenerative composition has regeneration and the homologous tissue that does not receive the administered regenerative composition also has regeneration.

In particular embodiments, one or more regenerative compositions are provided to a first tissue and/or organ in a body of an individual for the purpose of regeneration of a second tissue and/or organ that is not the same site of tissue and/or organ as the first tissue and/or organ but is of a similar type of tissue and/or organ as the first tissue and/or organ. Delivery of one or more regenerative compositions to a first tissue may result in regeneration of the first tissue and of a second tissue that is not the exact same location of tissue as the first tissue but that is of the same type of tissue as the first tissue.

The regenerative composition(s) may be administered either locally, such as administered at a tissue that is homologous to tissue that is to be regenerated, or systemically, such as when the tissue (or cell type) to be regenerated is located throughout the body, including blood cells for example. The administration may either be an injection, for example using a syringe to inject the regenerative composition(s), or the administration may be a delivery such as implantation of the regenerative composition at the site of delivery, merely as examples.

The tissue to be administered a regenerative composition or the tissue to be regenerated may be of any tissue type. For example, the tissue may be muscle tissue, connective tissue, epithelial tissue, endothelial tissue, nervous tissue, fat tissue, skin tissue, lung tissue, liver tissue, bladder tissue, kidney tissue, heart tissue, stomach tissue, intestinal tissue, spinal tissue, eye tissue, fibrous tissue, bone marrow, or a combination thereof. The tissue may be made up of any cell type including endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, interstitial kidney cells, or a combination thereof.

Reference to particular buffers, media, reagents, cells, culture conditions and the like, or to some subclass of same, is not intended to be limiting, but should be understood to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In specific embodiments, cells are cultured in a cell culture system that comprises a cell culture medium, such as in a culture vessel, in particular a cell culture medium supplemented with one or more substances suitable and determined for culturing the cells in a manner so as to endow ability to induce a regenerative effect that is acting systemically.

In specific embodiments, one can utilize regulators of genes to modify cells such as fibroblasts and/or stem cells to have therapeutic properties for use within the methods and regenerating compositions of the disclosure. A gene regulator may be a transcription factor, such as SOX2, NANOG, or OCT4. A reprogramming agent that causes dedifferentiation may also be utilized and may be utilized in combination or as an alternative to the gene regulator; examples of reprogramming agents include at least valproic acid, trichostatin A and lithium. In accordance with the disclosure presented herein, the embodiment of identifying a sufficient period of time to allow stable expression of the at least one gene regulator (for example, in the absence of a reprogramming agent) and a sufficient period of time in which the cell is to be maintained in culture conditions supporting the transformation of the desired cell is within the skill of those in the art. The sufficient or proper time period may vary according to various factors, including but not limited to, the particular type and epigenetic status of cells, for example the starting cell type and the desired cell type; the amount of starting material, for example the number of cells to be transformed; the amount and type of reprogramming agent(s); the gene regulator(s) (one or more agents that modulate gene expression); the culture conditions; and/or the presence of compounds that speed up reprogramming, for example compounds that increase cell cycle turnover, modify the epigenetic status, and/or enhance cell viability. In various embodiments, the sufficient period of time to allow a stable expression of the at least one gene regulator in absence of the reprogramming agent is about 1 day, about 2-4 days, about 4-7 days, about 1-2 weeks, about 2-3 weeks or about 3-4 weeks. In various embodiments the sufficient period of time in which the cells are to be maintained in culture conditions supporting the transformation of the desired cell and allow a stable expression of a plurality of secondary genes is about 1 day, about 2-4 days, about 4-7 days, or about 1-2 weeks, about 2-3 weeks, about 3-4 weeks, about 4-6 weeks or about 6-8 weeks. In specific embodiments, at the end of the transformation period, the number of transformed desired cells is substantially equivalent or even higher than an amount of cells a first type provided at the beginning.

Relating to the present disclosure, the standard growth conditions, as used herein may in some embodiments refer to culturing of cells at approximately 37° C., in a standard atmosphere comprising approximately 5% CO₂. Relative humidity may be maintained at about 100%. While foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

In some embodiments, a first muscle location is administered one or more regenerative compositions for the purpose of regeneration of a second, non-identical muscle location, including of the same muscle type. In other embodiments, a first joint location is administered one or more regenerative compositions for the purpose of regeneration of a second, non-identical muscle location. In particular cases, a first connective tissue location is administered one or more regenerative compositions for the purpose of regeneration of a second, non-identical connective tissue location. In particular cases, a first joint tissue location is administered one or more regenerative compositions for the purpose of regeneration of a second, non-identical joint tissue location. In some embodiments, a first epithelial tissue location is administered one or more regenerative compositions for the purpose of regeneration of a second, non-identical epithelial tissue location. In certain cases, a first endothelial tissue location is administered one or more regenerative compositions for the purpose of regeneration of a second, non-identical endothelial tissue location. In some aspects, a first nervous tissue location is administered one or more regenerative compositions for the purpose of regeneration of a second, non-identical nervous tissue location. In certain embodiments, a first fat tissue location is administered one or more regenerative compositions for the purpose of regeneration of a second, non-identical fat tissue location. In particular cases, a first skin tissue location is administered one or more regenerative compositions for the purpose of regeneration of a second, non-identical skin tissue location. In certain embodiments, a first lung tissue location is administered one or more regenerative compositions for the purpose of regeneration of a second, non-identical lung tissue location. In specific embodiments, a first liver tissue location is administered one or more regenerative compositions for the purpose of regeneration of a second, non-identical liver tissue location. In particular embodiments, a first bladder tissue location is administered one or more regenerative compositions for the purpose of regeneration of a second, non-identical bladder tissue location. In some cases, a first kidney tissue location is administered one or more regenerative compositions for the purpose of regeneration of a second, non-identical kidney tissue location. In specific embodiments, a first heart tissue location is administered one or more regenerative compositions for the purpose of regeneration of a second, non-identical heart tissue location. In specific cases, a first stomach tissue location is administered one or more regenerative compositions for the purpose of regeneration of a second, non-identical stomach tissue location. In some embodiments, a first intestinal tissue location is administered one or more regenerative compositions for the purpose of regeneration of a second, non-identical intestinal tissue location. In some cases, a first spinal tissue location is administered one or more regenerative compositions for the purpose of regeneration of a second, non-identical spinal tissue location. In certain aspects, a first eye tissue location is administered one or more regenerative compositions for the purpose of regeneration of a second, non-identical eye tissue location. In certain embodiments, a first fibrous tissue location is administered one or more regenerative compositions for the purpose of regeneration of a second, non-identical fibrous tissue location In specific aspects, a first bone tissue location is administered one or more regenerative compositions for the purpose of regeneration of a second, non-identical bone tissue location.

### III. Growth Factors and Platelet Rich Plasma

In particular embodiments, the regenerative composition(s) comprises at least one growth factor, and the growth factor may or may not be included in the composition with fibroblasts and/or stem cells. Growth factors, as used herein, refer to compounds, molecules, chemicals, proteins, peptides, nucleic acids, lipids, or vitamins that can stimulate the growth of a cell. In particular embodiments, the growth factor comprises a known molecule, peptide, and/or protein that can induce regeneration. Examples include AM, Ang, BMP, BDNF, EGF, Epo, FGF, GNDF, G-CSF, GM-CSF, GDF-9, HGF, HDGF, IGF, migration-stimulating factor, GDF-8, GDF-11, GDF-15, MGF, NGF, P1GF, PDGF, Tpo, TGF-alpha, TGF-beta, TNF-alpha, VEGF, a Wnt protein, an interleukin, a soluble receptor for IL-1alpha, IL-1beta, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12, 35 kDa alpha subunit, IL-12, 40 kDa beta subunit, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-24, IL-25, IL-26, IL-27B, IL-27-p28, IL-28A, IL-28B, IL-29, IL-30, IL-31, IL-32, IL-33, IL-34, IL-35, IL-36alpha, IL-36beta, IL-36gamma, an interferon (IFN), a soluble receptor for IFN-alpha, IFN-beta, IFN-gamma, IFN-lamdal, IFN-lamda2, IFN-lamda3, IFN-K, IFN-epsilon, IFN-kappa, IFN-tau, IFN-delta, IFN-zeta, IFN-omega, IFN-v, insulin, proinsulin, a receptor for insulin, leptin (LEP), or a combination thereof. Other growth factors may also be used. The growth factor may be produced in any method known in the art, such as by recombinant protein production, isolation from plasma or other cellular sources, peptide synthesis, for example. The amount of growth factor required for administration may be determined by one skilled in the art, and may be sufficient to induce regeneration.

In specific embodiments, the regenerative composition comprises platelet rich plasma that may or may not be included in the composition with fibroblasts and/or stem cells. Plasma may be obtained from any source, such as from sources autologous, allogenic, syngeneic, xenogeneic, or a combination thereof to the individual to be administered the regenerative composition. The plasma may be derived from any blood source, including peripheral blood or cord blood. The plasma may be manipulated to enrich for platelets, such that the platelet rich plasma has higher levels or a higher concentration of platelets or growth factors compared to normal, non-manipulated plasma. The concentration of platelets or growth factors in platelet rich plasma may be 2, 3, 4, 5, 6, 7, 8, 9, 10, 11, 12, 13, 14, 15, 16, 17, 18, 19, 20, or more times higher than the concentration of platelets or growth factors in normal, non-manipulated plasma. Any method to enrich plasma to become platelet rich plasma may be used, such as centrifugation. In some embodiments, the platelets or platelet rich plasma is further manipulated to obtain platelet lysate. Any method to obtain platelets prior to obtaining platelet lysate may be used, including centrifugation or apheresis, for example. Platelet lysate may be obtained from platelets or platelet rich lysate by any method, including freeze/thaw cycles, for example.

### IV. Stem Cells

In specific embodiments, the regenerative composition comprises one or more types of stem cells, including at least mesenchymal stem cells. “Mesenchymal stem cell” or “MSC” in some embodiments refers to cells that are (1) adherent to plastic, (2) express CD73, CD90, and CD105 antigens, or a combination thereof, while not expressing CD14, CD34, CD45, and HLA-DR, or a combination thereof, and (3) possess ability to differentiate to osteogenic, chondrogenic and adipogenic lineage [1, 2]. In some definitions, MSC include cells that are CD34 positive upon initial isolation from tissue (they may lose CD34 expression spontaneously) but are similar to cells described above phenotypically and functionally. MSCs may include cells that are isolated from tissues using cell surface markers selected from the group consisting of NGF-R, PDGF-R, EGF-R, IGF-R, CD29, CD49a, CD56, CD63, CD73, CD90, CD105, CD106, CD140b, CD146, CD271, MSCA-1, SSEA4, STRO-1, STRO-3 and a combination thereof (in specific embodiments, the cells are CD73-positive CD90-positive, and CD105 positive cells), and satisfy the International Society for Cellular Therapy (ISCT) criteria either before or after expansion. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. The MSC can be derived from any tissue including, but not limited to, bone marrow [3-7], adipose tissue [8, 9], amniotic fluid [10, 11], endometrium [12-15], trophoblast-associated tissues [16], human villous trophoblasts [17], cord blood [18], Wharton jelly [19-21], umbilical cord tissue [22], placenta [23], amniotic tissue [24-26], derived from pluripotent stem cells [27-31], peripheral blood, mobilized peripheral blood, umbilical cord blood, skeletal muscle, subepithelial umbilical cord tissue, menstrual blood, fallopian tube tissue, tooth, or a combination thereof.

Furthermore, as used herein, in some contexts, MSCs include cells described in the art as bone marrow stromal stem cells (BMSSC) [32], marrow-isolated adult multipotent inducible cells (MIAMI) cells [33, 34], multipotent adult progenitor cells (MAPC) [35-38], MultiStem®, Prochymal [39-43], remestemcel-L [44], Mesenchymal Precursor Cells (MPCs) [45], Dental Pulp Stem Cells (DPSCs) [46], PLX cells [47], Ixmyelocel-T [48], NurOwn™ [49], Stemedyne™-MSC, Stempeucel® [50, 51], HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs) [52].

The MSC may be expanded and utilized by administration themselves, or may be cultured in a growth media in order to obtain conditioned media. The growth medium may refer to a medium sufficient for the culturing of umbilicus-derived cells. In particular, one medium for the culturing of the cells of the disclosure herein comprises Dulbecco's Modified Essential Media (also abbreviated DMEM herein). In some embodiments, the medium is DMEM-low glucose (also DMEM-LG herein). The DMEM-low glucose may be supplemented with a supplements comprising 1%, 5%, 10%, 15%, or 20% (v/v) fetal bovine serum; an antibiotics/antimycotics solution, for example penicillin at a suitable concentration, which may be about 100 units/milliliter; streptomycin at a suitable concentration, which may be about 100 milligrams/milliliter; amphotericin B at a suitable concentration, which may be about 0.25 micrograms/milliliter; 2-mercaptoethanol at a suitable concentration, which may be 0.001% (v/v); or a combination thereof.

Mesenchymal stem cells may be derived from the embryonal mesoderm or may be isolated from adult bone marrow and other adult tissues including peripheral blood, adipose tissue, mobilized peripheral blood, skeletal muscle tissue, endometrial tissue, menstrual blood, fallopian tube tissue, for example. They may be differentiated to form any tissue including muscle, bone, cartilage, fat, marrow stroma, tendon, for example. Mesoderm also may differentiate into visceral mesoderm which may give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (stem cells that are SH2+, SH4+, CD29+, CD44+, CD71,+CD90+, CD106+, CD120a+, CD124+, CD14-, CD34-, and/or CD45)). The present disclosure encompasses the use of various mesenchymal stem cells.

In particular embodiments, MSC are generated from tissues including umbilical cord tissue, umbilical cord blood, Wharton's jelly, supepithelial umbilical cord, or a combination thereof. Means of generating umbilical cord tissue MSC have been previously disclosed and are incorporated by reference [18, 21, 53-57]. The term “umbilical tissue derived cells (UTC)” refers, for example, to cells as described in U.S. Pat. No. 7,510,873, U.S. Pat. No. 7,413,734, U.S. Pat. No. 7,524,489, and U.S. Pat. No. 7,560,276. The UTC may be of any mammalian origin including human, rat, primate, porcine and the like, for example. In particular embodiments of the disclosure, the UTC are derived from human umbilicus. Umbilicus-derived cells have reduced expression of genes, relative to a human cell such as a fibroblast, a mesenchymal stem cell, or an iliac crest bone marrow cell, for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744;cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle).

In addition, these isolated human umbilicus-derived cells express at least one gene for at least one of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell such as a fibroblast, a mesenchymal stem cell, an iliac crest bone marrow cell, or placenta-derived cell. The cells may also secrete factors or proteins including MCP-1, MIP1beta, IL-6, IL-8, GCP-2, HGF, KGF, FGF, HB-EGF, BDNF, TPO, RANTES, TIMP1, or a combination thereof. The cells may express specific markers including, for example, TRA1-60, TRA1-81, SSEA3, SSEA4, NANOG, or a combination thereof. The cells are capable of self-renewal and expansion in culture, and have the potential to differentiate into cells of other phenotypes.

Methods of deriving cord tissue mesenchymal stem cells from human umbilical tissue are provided. The cells may be capable of self-renewal and expansion in culture, and may have the potential to differentiate into cells of other phenotypes. The method comprises (a) obtaining human umbilical tissue; (b) removing substantially all of blood to yield a substantially blood-free umbilical tissue, such that no blood that is capable of self-renewal and expansion in culture is present, (c) dissociating the tissue by mechanical or enzymatic treatment, or both, (d) resuspending the tissue in a culture medium, and (e) providing growth conditions that allow for the growth of a human umbilicus-derived cell capable of self-renewal and expansion in culture and having the potential to differentiate into cells of other phenotypes. Growth conditions may include culture in media such as RPMI, DMEM, EMEM, or customized media.

In specific embodiments, cells may be obtained from umbilical cord. Tissue may be obtained from any completed pregnancy, term or less than term, whether delivered vaginally, or through other routes, for example surgical Cesarean section. Obtaining tissue from tissue banks is also considered within the scope of the present disclosure. The tissue may be rendered substantially free of blood by any means known in the art. For example, the blood may be physically removed by washing, rinsing, and diluting and the like, before or after bulk blood removal for example by suctioning or draining. Other means of obtaining a tissue substantially free of blood cells may include enzymatic or chemical treatment. Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In specific embodiments, any isolation procedure for cells utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan, ranging from weakly digestive (e.g. deoxyribonucleases and the neutral protease, dispase) to strongly digestive (e.g. papain and trypsin), and such enzymes are available commercially. A non-exhaustive list of enzymes compatible herewith includes mucolytic enzyme activities, metalloproteases, neutral proteases, serine proteases (such as trypsin, chymotrypsin, or elastase), and deoxyribonucleases. Enzyme activities that may be useful include those selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Some embodiments include methods that involve enzymatic treatment with, for example, collagenase and dispase, or collagenase, dispase, and hyaluronidase. Methods are provided wherein a mixture of collagenase and the neutral protease dispase are used in the dissociating step. Methods may be use that employ digestion in the presence of at least one collagenase from Clostridium histolyticum, and either of the protease activities, dispase and/or thermolysin. Methods employing digestion with both collagenase and dispase enzyme activities may also be used.

Methods may be used that include digestion with a hyaluronidase activity in addition to collagenase and dispase activities. The skilled artisan will appreciate that many such enzyme treatments are known in the art for isolating cells from various tissue sources. For example, the LIBERASE™ BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the disclosure. Enzyme treatments may be 0.5, 1, 1.5, or 2 hours long or longer. In other embodiments, the tissue is incubated at approximately 37° C. during the enzyme treatment of the dissociation step. Diluting the digest may also improve yields of cells as cells may be trapped within a viscous digest. While the use of enzyme is presently preferred, it is not required for isolation methods as provided herein. Methods based on mechanical separation alone may be successful in isolating the instant cells from the umbilicus as discussed above.

The cells may be resuspended after the tissue is dissociated into any culture medium as discussed herein above. Cells may be resuspended following a centrifugation step, which is used to separate out the cells from tissue or other debris. Resuspension may involve mechanical methods of resuspending, or simply the addition of culture medium to the cells. Providing the growth conditions allows for a wide range of options as to culture medium, supplements, atmospheric conditions, and relative humidity for the cells. The culture temperature may be approximately 37° C., however the temperature may range from about 35° C. to about 39° C. depending on the other culture conditions and desired use of the cells or culture.

In particular embodiments, methods are employed in that cells require no exogenous growth factors, except, in at least some cases, growth factors are available in the supplemental serum provided with the growth medium. Also provided herein are methods of deriving umbilical cells capable of expansion in the absence of particular growth factors. The methods are similar to the method above, however they may require that the particular growth factors (for which the cells have no requirement) be absent in the culture medium in which the cells are ultimately resuspended and grown in. In this sense, the method is selective for those cells capable of division in the absence of the particular growth factors. Particular cells, in some embodiments, are capable of growth and expansion in chemically-defined growth media with no serum added. In such cases, the cells may require certain growth factors, which can be added to the medium to support and sustain the cells. Factors that may be added for growth on serum-free media may comprise one or more of FGF, EGF, IGF, and PDGF. In some embodiments, two, three or all four of the factors are add to serum free or chemically defined media. In specific embodiments, leukemia inhibitory factor (LIF) is added to serum-free medium to support or improve growth of the cells.

Also provided are methods wherein the cells can expand in the presence of about 5% to about 20% oxygen in their atmosphere. Methods to obtain cells may also require that cells be cultured in the presence of L-valine. After a cell is obtained, its need for L-valine can be tested and confirmed by growing on D-valine containing medium that lacks the L-isomer.

In some embodiments, the umbilicus-derived cells, including cells from umbilical cord blood, umbilical cord tissue, Wharton's jelly, and/or subepithelial umbilical cord, can undergo at least 1, 2, 3, 4, 5, 10, 15, 20, 25, 30, 35, or 200 doublings prior to reaching a senescent state. Cell may maintain a normal karyotype throughout passaging. Methods for deriving cells capable of doubling to reach 1×10¹⁴ cells or more are provided. Methods may be used which derive cells that can double sufficiently to produce at least about 1×10¹⁴, 1×10¹⁵, 1×10¹⁶, or 1×10¹⁷ or more cells when seeded at from about 1×10³ to about 1×10⁶ cells/cm² in culture. In some embodiments, cell numbers are produced within 80, 70, or 60 days or less. In particular embodiments, umbilicus-derived mesenchymal stem cells are isolated and expanded, and possess one or more markers or proteins selected from the group consisting of CD9, CD10, CD13, C29, CD44, CD73, CD90, CD105, CD146, CD141, CD166, SSEA4, PDGFr-alpha, HLA-A,B,C, SOX2, OCT4, and a combination thereof. In addition, the cells do not produce one or more of the markers selected from the group consisting of CD14, CD31, CD34, CD45, CD79, CD80, CD86, CD106, CD117, CD141, HLA-DR,DP, DQ, STRO-1, and a combination thereof.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA , washed with DPBS +2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody . Confluent MSC in 175 cm2 flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 mL of M199 +1% human serum albumin (HSA; American Red Cross, Washington D.C., USA). MSCs harvested from each 10-flask set were stored for up to 4 h at 4° C. and combined at the end of the harvest. A total of 2-10′ 10⁶ MSC/kg were resuspended in M199 +1% HSA and centrifuged at 460 g for 10 min at 20° C. Cell pellets were resuspended in fresh M199 +1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, IL, USA) freezing bags using a rate controlled freezer at a final concentration of 10% DMSO (Research Industries, Salt Lake City, UT, USA) and 5% HSA. On the day of infusion cryopreserved units were thawed at the bedside in a 37° C. water bath and transferred into 60 mL syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In specific embodiments, MSC are generated according to protocols previously utilized for treatment of patients utilizing bone marrow derived MSC. The bone marrow cells may express specific markers including LFA-3, ICAM-1, PECAM-1, P-selectin, L-selectin, CD49b/CD29, CD49c/CD29, CD49d/CD29, CD29, CD18, CD61, 6-19, thrombomodulin, telomerase, CD10, CD13, integrin beta, CD34, CD56, CD117, or a combination thereof. In some embodiments, the bone marrow derived MSCs may not express CD10, CD2, CD3, CDS, CD14, CD16, CD19, C31, CD33, CD45, CD64, and/or DRII. In some embodiments, the bone marrow derived cells comprise, at least in part, MSC progenitor cells. The progenitor cells may express STRO-1, TKY-1, CD146, STRO-2, and/or VCAM-1 and may lack expression of CBFA-1, collagen type II, PPAR.gamma2, osteopontin, osteocalcin, parathyroid hormone receptor, leptin, H-ALBP, aggrecan, Ki67, glycophorin A, or a combination thereof.

Bone marrow may be aspirated from the posterior iliac crest to generate approximately between 10-30 mL, which is collected into containers and may be transferred to a clean room. The containters may be sodium heparin containing tubes. Good manufacturing practices (GMP) may be used. While the procedure is performed the individual may be provided local anesthesia, with or without sedation. Bone marrow cells may be washed with a washing solution such as Dulbecco's phosphate-buffered saline (DPBS), RPMI, or PBS supplemented with autologous patient plasma, for example. The cells may be layered on to Percoll, or similar solution, at a concentration of approximately 1-2×10⁷ cells/mL. Subsequently the cells may be centrifuged at approximately 700-1100×g for approximately 30 min or a time period sufficient to achieve separation of mononuclear cells from debris and erythrocytes. Said cells may then be washed with a solution such as PBS for example and plated at a density of approximately 1×10⁶ cells per mL in a suitable culture container, such as a 175 cm² tissue culture flasks, in culture medium such as DMEM with 10% FCS. Said flasks may subsequently being loaded with a minimum of 30 million bone marrow mononuclear cells. The MSCs are allowed to adhere for at least 24 hours. MSCs may be allowed to adhere for 72 h or more. The MSC may then be subjected to media changes every 3-4 days. Adherent cells may then be removed using a suitable method including with 0.05% trypsin-EDTA and replated at a density of approximately 1×10⁶ per 175 cm². Said bone marrow MSC may be administered intravenously, or in a particular embodiment, intrathecally in an individual in need thereof, including at least one suffering radiation associated neurodegenerative manifestations. Although doses may be determined by one of skill in the art, and are dependent on various characteristics of the individual being administered the cells, intravenous administration may be performed at cell numbers ranging from 1-10 million MSC per kilogram of individual's measured weight. The administration may be performed at a cell number between approximately 2-5 million cells per kilogram of the individual's measured weight.

In specific embodiments, the regenerative composition comprises hematopoietic stem cells, which may be are CD34+ cells isolated from the peripheral blood, bone marrow, or umbilical cord blood. The hematopoietic stem cell may express c-kit, Sca-1, CD34, CD133, or a combination thereof. The hematopoietic stem cell may lack the expression of C38 or other lineage markers. The hematopoietic stem cells may be derived from the blood system of mammalian animals, include but not limited to human, mouse, rat, and these hematopoietic stem cells may be harvested by isolating from the blood or tissue organs in mammalian animals. Examples of sources for hematopoietic stem cells include peripheral blood, mobilized peripheral blood, bone marrow, cord blood, adipose stromal vascular fraction, derived from progenitor cells, or a combination thereof. Hematopoietic stem cells may be harvested from a donor by any known methods in the art. For example, U.S. Pub. 2013/0149286 details procedures for obtaining and purifying stem cells from mammalian cadavers. Stem cells may be harvested from a human by bone marrow harvest or peripheral blood stem cell harvest, both of which are well known techniques in the art. After stem cells have been obtained from the source, such as from certain tissues of the donor, they may be cultured using stem cell expansion techniques. Stem cell expansion techniques are disclosed in U.S. Pat. No. 6,326,198 to Emerson et al., entitled “Methods and compositions for the ex vivo replication of stem cells, for the optimization of hematopoietic progenitor cell cultures, and for increasing the metabolism, GM-CSF secretion and/or IL-6 secretion of human stromal cells,” issued Dec. 4, 2001; U.S. Pat. No. 6,338,942 to Kraus et al., entitled “Selective expansion of target cell populations,” issued Jan. 15, 2002; and U.S. Pat. No. 6,335,195 to Rodgers et al., entitled “Method for promoting hematopoietic and cell proliferation and differentiation,” issued Jan. 1, 2002, which are hereby incorporated by reference in their entireties. In some embodiments, stem cells obtained from the donor are cultured in order to expand the population of stem cells. In specific embodiments, stem cells collected from donor sources are not expanded using such techniques. Standard methods can be used to cyropreserve the stem cells.

In some embodiments of the disclosure, where there are risks associated with particular types of stem cells, for example, pluripotent stem cells, said stem cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval. A wide variety of materials may be used in various embodiments for microencapsulation of stem cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. Techniques for microencapsulation of cells that may be used for administration of stem cells are known to those of skill in the art and are described, for example, in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of stem cells. Certain embodiments incorporate stem cells into a polymer, such as a biopolymer or synthetic polymer. Examples of biopolymers include, but are not limited to, fibronectin, fibrin, fibrinogen, thrombin, collagen, and proteoglycans. Other factors, such as the cytokines discussed above, can also be incorporated into the polymer. In other embodiments of the disclosure, stem cells may be incorporated in the interstices of a three-dimensional gel. A large polymer or gel may be surgically implanted. A polymer or gel that can be formulated in small enough particles or fibers can be administered by other common, more convenient, non-surgical routes.

In some embodiments, the regenerative composition is at least one skeletal muscle derived mesenchymal stem cell. The skeletal muscle derived mesenchymal stem cell may express CD13, CD34, CD56, CD117, or a combination thereof and may lack expression of CD10, CD2, CDS, CD14, CD19, CD33, CD45, and/or DRII.

In some embodiments of the disclosure, mesenchymal stem cells are cultured with substances capable of maintaining said mesenchymal stem cells in an immature state, and/or maintaining high expression of genes/mitochondria necessary to allow for generation of a systemically acting regenerative effect. In some embodiments, the systemic effect is targeted towards inducing regeneration in tissues that are homologous to tissues treated with the regenerative composition. The substances are selected from the group consisting of reversin, cord blood serum, lithium, a GSK-3 inhibitor, resveratrol, pterostilbene, selenium, a selenium-containing compound, EGCG ((−)-epigallocatechin-3-gallate), valproic acid and salts of valproic acid, in particular sodium valproate. In specific embodiments of the present disclosure, a concentration of reversin from 0.5 to 10 μM, preferably of 1 μM is added to the mesenchymal stem cell culture. Specific embodiments of the present disclosure foresee to use resveratrol in a concentration of 10 to 100 μM, preferably 50 μM. The present disclosure provides methods that may use selenium or a selenium containing compound in a concentration from 0.05 to 0.5 μM, preferably of 0.1 μM. In specific embodiments, cord blood serum is added at a concentration of 0.1%- 20% volume to the volume of tissue culture media. In some embodiments, the present disclosure foresees to use EGCG in a concentration from approximately 0.001 to 0.1 μM, preferably at approximately 0.01 μM. In particular embodiments, the present disclosure foresees to use valproic acid or sodium valproate in a concentration from 1 to 10 μM, and in particular embodiments, at 5 μM. In some embodiments, mesenchymal stem cells are dedifferentiated to possess higher expression of regenerative genes. The dedifferentiated may be achieved by cytoplasmic transfer, transfection of cytoplasm, or cell fusion with a stem cell possessing a higher level of immaturity, wherein the stem cells include pluripotent stem cells. In such culture/coculture procedures, the cell culture medium comprises, optionally in combination with one or more of the substances specified above, at least one transient proteolysis inhibitor. The use of at least one proteolysis inhibitor in the cell culture medium of the present disclosure increases the time the reprogramming proteins derived from the mRNA or any endogenous genes will be present in the cells and thus facilitates, in an even more improved way, the reprogramming by the transfected mRNA.

In some embodiments of the disclosure, the regenerative composition is at least one pluripotent stem cell. The pluripotent stem cell may be of any origin or type including stem cells, parthenogenic derived stem cells, inducible pluripotent stem cells, somatic cell nuclear transfer derived stem cells, cytoplasmic transfer derived stem cells, stimulus-triggered acquisition of pluripotency, or a combination thereof. Any method for obtaining pluripotent stem cells may be used.

### V. Inhibitors

The present disclosure uses in particular embodiments one or more inhibitors, for example to generate cells with regenerative activity, or the cells can be used to induce regenerative abscobal effect. Examples of inhibitors include a proteolysis inhibitor (transient or not), a protease inhibitor, a proteasome inhibitor and/or a lysosome inhibitor. In an embodiment the proteosome inhibitor is selected from the group consisting of MG132, TMC-95A, TS-341 and MG262. In particular embodiments, the protease inhibitor is selected from the group consisting of aprotinin, G-64, leupeptine-hemisulfat, and a combination thereof. In specific embodiments, the lysosomal inhibitor is ammonium chloride. The present disclosure also encompasses a cell culture medium comprising at least one transient inhibitor of mRNA degradation. The use of a transient inhibitor of mRNA degradation increases the half-life of the reprogramming factors as well. Particular embodiments of the present disclosure allow for a condition suitable to allow translation of the transfected reprogramming mRNA molecules in the cells is an oxygen content in the cell culture medium from 0.5 to 21%. More particular, and without wishing to be bound to the theory, oxygen is used to further induce or increase Oct4 by triggering Oct4 via HIF-1α, in these situations concentrations of oxygen lower than atmospheric concentration are used, and can be ranging from 0.1% to 10%. In specific embodiments, conditions may be suitable to support reprogramming of the cells by the mRNA molecules in the cells are selected; more particularly, these conditions require a temperature from 30 to 38° C., preferably from 31 to 37° C., most preferably from 32 to 36° C. The glucose content of the medium may be below 4.6 g/L, 4.5 g/L, 4 g/L, 3 g/L, 2 g/L, or at or below approximately 1 g/L. DMEM media containing 1 g/L glucose are commercially available as “DMEM low glucose” from companies such as PAA, Omega Scientific, Perbio and Biosera. More particular, and without wishing to be bound to the theory, high glucose conditions adversely support aging of cells, such as by methylation or changes in epigenetics, for example, in vitro which may render the reprogramming difficult. In specific embodiments of the present disclosure, the cell culture medium contains glucose in a concentration from 0.1 g/L to 4.6 g/L, 0.5 g/L to 4.5 g/L, or 1 g/L to 4 g/L.

In some embodiments of the disclosure, enhancement of abscopal effect is achieved by administration of one or more epigenetic modifiers that enhance either the potency of the regenerative composition or the ability of endogenous stem cells to respond to signals systemically secreted by regenerative composition at a distant location. The regenerative composition used in particular embodiments may be enhanced by the epigenetic acting composition. The epigenetic acting composition may be delivered concurrently with the regenerative composition or prior to administration of the regenerative composition or after administration of the regenerative composition. The epigenetic acting composition may be administered at the same site as the regenerative composition or at a different anatomical site, including potentially the site that is to be regenerated.

The epigenetic modifiers include, by way of example, epigenetic modifiers such as DNA demethylating agents, histone deacetylase (HDAC) inhibitors, histone modifiers, and cell cycle manipulation and pluripotent or tissue specific promoting agents such as helper cells which promote growth or production of pluripotent cells, growth factors, hormones, and bioactive molecules. Examples of DNA demethylating agents include, but are not limited to, 5-azacytidine (5-aza), N-methyl-N′-nitro-N-nitrosoguanidine, temozolomide, procarbazine, etc. Examples of methylation inhibiting agents include decitabine, 5-azacytidine, hydralazine, procainamide, mitoxantrone, zebularine, 5-fluorodeoxycytidine, 5-fluorocytidine, anti-sense oligonucleotides against DNA methyltransferase, or other inhibitors of enzymes involved in the methylation of DNA.

Examples of HDAC inhibitors include, but are not limited to, hydroxamic acids, cyclic peptides, benzamides, short-chain fatty acids, and depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include, but are not limited to, MS-27-275. Examples of short-chain fatty acids include, but are not limited to, butyrates (e.g., butyric acid, phenylbutyrate (PB) and CI-994 (acetyldinaline). Examples of histone modifiers include, but are not limited to, PARP, the human enhancer of zeste, valproic acid, and trichostatin A. In some embodiments, the particular modifiers utilized in cell culture media comprise trichostatin A, valproic acid, zebularine and/or 5-aza, in order to facilitate RNA transfection and dedifferentiation of the RNA-comprising target cells into pluripotent cells. Target cells into which RNA is introduced are cultured for a sufficient time in media that promotes RNA transfection until dedifferentiated cells (pluripotent) cells are obtained. In some embodiments, this methodology may be combined with other methods and treatments involved in regulating or altering the epigenetic status of the recipient or target cell, such as the exposure to DNA and/or histone demethylating agents, histone deacetylase inhibitors, and/or histone modifiers. This disclosure therefore describes, in some embodiments, methods of changing the fate or phenotype of cells. By using epigenetic modifying compositions, the disclosed methods may dedifferentiate or transdifferentiate cells.

### VI. Exosomes

Exosomes, also referred to as “particles” may comprise vesicles or a flattened spheres comprised of a lipid bilayer. The particles may comprise diameters of 2-200 nm, 5-200 nm, 10-200 nm, 15-200 nm, 20-200 nm, 30-200 nm, 40-200 nm, 50-200 nm, 60-200 nm, 70-200 nm, 80-200 nm, 90-200 nm, 100-200 nm, 110-200 nm, 120-200 nm, 130-200 nm, 140-200 nm, 150-200 nm, 160-200 nm, 170-200 nm, 180-200 nm, 190-200 nm, 2-150 nm, 5-150 nm, 10-150 nm, 15-150 nm, 20-150 nm, 30-150 nm, 40-150 nm, 50-150 nm, 60-150 nm, 70-150 nm, 80-150 nm, 90-150 nm, 100-150 nm, 110-150 nm, 120-150 nm, 130-150 nm, 140-150 nm, 2-100 nm, 5-100 nm, 10-100 nm, 15-100 nm, 20-100 nm, 30-100 nm, 40-100 nm, 50-100 nm, 60-100 nm, 70-100 nm, 80-100 nm, 90-100 nm, 2-50 nm, 5-50 nm, 10-50 nm, 15-50 nm, 20-50 nm, 30-50 nm, 40-50 nm, or any range in between. Any method to determine the size of an exosome may be used including dynamic light scattering or nanoparticle tracking analysis, for example. The particles may be formed by inward budding of the endosomal membrane. The particles may have a density of about 1.13-1.19 g/mL and may float on sucrose gradients. The particles may be enriched in cholesterol and sphingomyelin, markers such as CD9, CD63, CD81, ANXA2, ENO1, HSP9OAA1, EEF1A1, YWHAE, SDCBP, PDCD6IP, ALB, YWHAZ, EEF2, ACTG1, LDHA, HSP90AB1, ALDOA, MSN, ANXAS, PGK1, CFL1, and lipid raft markers such as GM1, GM3, flotillin and the src protein kinase Lyn. The particles may be comprised of at least one lipid selected from the group consisting of phospholipids, phosphatidyl serine, phosphatidyl inositol, phosphatidyl choline, sphingomyelin, ceramides, glycolipid, cerebroside, steroids, cholesterol, and a combination thereof. The particles may comprise at least one lipid raft. The particles may comprise one or more proteins present in mesenchymal stem cells, or any cell of the present disclosure, or mesenchymal stem cell conditioned medium (MSC-CM), or any media of the present disclosure, such as a protein characteristic or specific to the MSC or MSC-CM, for example. They may comprise RNA, for example miRNA. Said particles may possess one or more genes or gene products found in MSCs, or any cell of the present disclosure, or medium which is conditioned by culture of MSCs, or any medium of the present disclosure. The particle may comprise molecules secreted by the MSC, or any cell of the present disclosure. Such a particle, and combinations of any of the molecules comprised therein, including in particular proteins or polypeptides, may be used to supplement the activity of, or in place of, the MSCs, or any cell of the present disclosure, or medium conditioned by the MSCs, or any cell of the present disclosure, for the purpose of, for example, treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular embodiments, the particle may comprise one or more tetraspanins. The particles may comprise mRNA and/or microRNA. The particle may be used for any of the therapeutic purposes that the MSC, or any cell of the present disclosure, or MSC-CM, or any media of the present disclosure, may be put to use.

In particular embodiments, exosomes, or particles may be produced by culturing mesenchymal stem cells or fibroblasts, or any cell, including regenerative cells, of the present disclosure. The cells may be derived from any tissue source including foreskin, adipose tissue, placenta, ear lobe, omentum, Wharton's jelly, or a combination thereof. The mesenchymal stem cells, for example, may comprise human umbilical tissue derived cells which possess markers selected from the group consisting of CD90, CD73, CD105, and a combination thereof. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/mL FGF2. The concentration of PDGF AB may be about 5 ng/mL. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days.

The conditioned medium comprising exosomes may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6×40 mm or a TSK gel G4000 SWXL, 7.8×300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 mL/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rh of particles in this peak is about 45-55 nm. Such fractions may comprise mesenchymal stem cell particles, for example, such as exosomes.

In some embodiments of the disclosure, generation of a systemically acting regenerative effect is performed by administration of cellular lysate from regenerative cells. The regenerative cells may be mesenchymal stem cells, for example, or any cell of the present disclosure. In some embodiments the mesenchymal stem cells are derived from the umbilical cord, but may be of any tissue type. Derivation of mesenchymal stem cells from umbilical cord/Wharton's Jelly for clinical applications are described in the art and incorporated by reference [58-66]. For some embodiments of the disclosure, xenogeneic free media may be used to grow mesenchymal stem cells, or any cell of the present disclosure, to reduce the possibility of sensitization from components such as fetal calf serum [22, 67-73]. In some embodiments of the disclosure, mesenchymal stem cells are pretreated using ways of enhancing regenerative activity, said means include treatment with histone deacetylase inhibitors such as valproic acid, GSK-3 inhibitors such as lithium [74-79], culture under hypoxia, and treatment with carbon monoxide [80]. In some embodiments fibroblasts are utilized as an alternative for stem cell administration.

In some embodiments, mesenchymal stem cells, for example, may be synchronized in G2 by incubating the cells in the presence of aphidicolin to arrest them in S phase and then washing the cells three times by repeated centrifugation and resuspension in phosphate buffered saline (PBS), as described herein. The cells may then be incubated for a length of time sufficient for cells to enter G2 phase. For example, cells with a doubling time of approximately 24 hours, may be incubated for between 6 and 12 hours to allow them to enter G2 phase. For cells with shorter or longer doubling times, the incubation time may be adjusted accordingly. In some embodiments of the disclosure, mesenchymal stem cells may be synchronized in mitosis by incubating them in 0.5 μg/mL nocodazole for 17-20 hours, and the mitotic cells are detached by vigorous shaking. The detached G1 phase doublets may be discarded, or they may be allowed to remain with the mitotic cells which constitute the majority (over 80%) of the detached cells. The harvested detached cells are centrifuged at 500 g for 10 minutes in a 10 mL conical tube at 4° C. Synchronized or unsynchronized cells may be harvested using standard methods and washed by centrifugation at 500 g for 10 minutes in a 10 mL conical tube at 4° C. The supernatant is discarded, and the cell pellet is resuspended in a total volume of 50 mL of cold PBS. The cells are centrifuged at 500 g for 10 minutes at 4° C. This washing step is repeated, and the cell pellet is resuspended in approximately 20 volumes of ice-cold interphase cell lysis buffer (20 mM Hepes, pH 8.2, 5 mM MgCl2, 1 mM DTT, 10 pM aprotinin, 10 pM leupeptin, 10 pM pepstatin A, 10 pM soybean trypsin inhibitor, 100 pM PMSF, and optionally 20 pg/mL cytochalasin B). The cells are sedimented by centrifugation at 800 g for 10 minutes at 4° C. The supernatant is discarded, and the cell pellet is carefully resuspended in no more than one volume of interphase cell lysis buffer. The cells are incubated on ice for one hour to allow swelling of the cells. The cells are then lysed by either sonication using a tip sonicator or Dounce homogenization using a glass mortar and pestle. Cell lysis is performed until at least 90% of the cells and nuclei are lysed, which may be assessed using phase contrast microscopy. Duration and power of sonication required to lyse at least 90% of the cells and nuclei may vary depending on the type of cell used to prepare the extract.

In some embodiments, the cell lysate is placed in a 1.5-mL centrifuge tube and centrifuged at a speed between 10,000-15,000 g for 15 minutes at 4° C. using a table top centrifuge. The tubes are removed from the centrifuge and immediately placed on ice. The supernatant is carefully collected using a 200 μL pipette tip, and the supernatant from several tubes is pooled and placed on ice. This supernatant is the cytoplasmic extract. This cell extract may be aliquoted into 20 pL volumes of extract per tube on ice and immediately flash-frozen on liquid nitrogen and stored at 80° C. until use. Alternatively, the cell extract is placed in an ultracentrifuge tube on ice (e.g., fitted for an SW55 Ti rotor; Beckman). If necessary, the tube is overlaid with mineral oil to the top. The extract is centrifuged at 200,000 g for three hours at 4° C. to sediment membrane vesicles contained in the cytoplasmic extract. At the end of centrifugation, the oil is discarded. The supernatant is carefully collected, pooled if necessary, and placed in a cold 1.5 mL tube on ice.

In particular embodiments, mesenchymal stem cell, or any cell of the present disclosure, lysate is generated by rinsing cells 3-4 times with PBS, and culture medium, such as alpha-MEM or DMEM/F12 (Gibco) is added without additives or serum. 12-24 hours later, the cells are washed twice with PBS and harvested, such as by scraping with a rubber policeman and collected in a 50 mL Falcon tube (Becton Dickinson). Then cells are washed and resuspended in ice-cold cell lysis buffer (20 mM HEPES, pH 8.2, 50 mM NaCl, 5 mM MgCl.sub.2, 1 mM dithiothreitol and a protease inhibitor cocktail), sedimented at 400 g and resuspended in one volume of cell lysis buffer. Cells are sonicated on ice in 200 μL aliquots using a sonicator fitted with a 2-mm diameter probe until all cells and nuclei are lysed, as can be judged by phase contrast microscopy. The lysate is centrifuged at a speed between 10,000-14,000 g for 15-30 minutes at 4° C. to pellet the coarse material and any potentially remaining non-lysed cell. The supernatant is aliquoted, frozen and stored in liquid nitrogen or immediately used. Protein concentration of the extract is analyzed by Bradford assay, pH is adjusted to around 7.0.+-.0.4 and osmolarity is adjusted to −300 mOsm prior to use, in necessary, such as by diluting with water.

In addition to cell lysate, conditioned media from cells may be utilized. Both cell lysate and conditioned media may be administered intranasally through an aerosolation means, or may be administered orally, intravenously, subcutaneously, intrarectally, intramuscularly, intra-articularly, or sublingually.

Conditioned media may be generated in order to concentrate secreted factors, or may be utilized as a source of exosomes. In some embodiments, exosomes are concentrated by means of ultracentrifugation, chromatography, or based on adhesion to substrates.

### VI. Fibroblasts

Embodiments of the present disclosure are directed to systems and methods for the use of fibroblast cells from autologous, allogeneic, syngeneic, and/or xenogeneic sources. Methods and compositions of the disclosure encompass certain manipulated cells for the treatment of inflammatory, autoimmune, or other degenerative conditions. In particular, the cells include at least fibroblasts of any kind. Means of manipulation of fibroblasts are disclosed, as well as fibroblasts of different tissue origins, which actively inhibit degenerative processes. In one embodiment of the disclosure, fibroblasts are utilized for their ability to inhibit immune responses and also utilized as a cellular therapy for prevention and/or treatment of degenerative conditions. In at least one embodiment, fibroblasts are treated with one or more particular agents and/or conditions to be able to directly or indirectly treat degenerative processes. In particular embodiments, the agent comprises interferon gamma and/or platelet rich plasma, and in some cases at least interferon gamma and/or platelet rich plasma (and/or platelet rich lysate) can endow the ability of the fibroblasts to directly or indirectly actively suppress immune responses. Fibroblasts cultured under these conditions are administered into individuals suffering from autoimmune or inflammatory or other degenerative disorders or at risk thereof. The route of administration, dosage and frequency is determined as a function of the disease process, as well as stage of the disease, and can be optimized per routine practices in medicine.

In some embodiments of the disclosure, the regenerative composition is at least one fibroblast cell. The fibroblast may be of any origin and be from any tissue type including, for example, foreskin, adipose, placenta, ear lobe, omentum, Wharton's jelly, or a combination thereof. Methods to isolate fibroblasts are well known in the art, and any such method may be used to obtain fibroblasts. In particular embodiments, the fibroblast expresses at least one marker, which may be, for example, NANOG, OCT-4, SSEA-4, CD90, CD105, CD73, stem cell factor receptor, or a combination thereof. The fibroblast may be cultured prior to use in the methods for regeneration. Any suitable method and culture medium for culturing fibroblasts may be used, including culture methods and media above. In particular embodiments, the fibroblast may be used to generate exosomes, wherein the exosomes are used as the regenerative composition in particular methods disclosed herein. In particular embodiments, the fibroblast may be used to generate apoptotic vesicles, wherein the apoptotic vesicles may be used as the regenerative composition. In specific embodiments, the fibroblast may be used to obtain or derive miRNAs, wherein the miRNAs derived or obtained from the fibroblast may be used as the regenerative composition.

In particular embodiments, fibroblasts (whether allogeneic, autologous, or xenogeneic) are administered to an individual in a non-manipulated manner (for example, without prior exposure to one or more particular agents, such as interferon gamma) but selected from sources naturally characterized by immune modulatory activity, such as placental fibroblasts or adipose tissue-associated fibroblasts, for example. In other embodiments of the disclosure, any fibroblasts are cultured under conditions capable of inducing retro-differentiation so as to endow an immature phenotype for the fibroblasts, wherein the immature phenotype correlates with enhanced anti-inflammatory and/or immune modulatory potential. For example, fibroblasts may be cultured in the presence of one or more histone deacetylase inhibitors, such as valproic acid (Moon et al., 2008; Huang et al., 2011). In addition to HDAC inhibitors, other means of inducing dedifferentiation of the fibroblasts may also be utilized in the context of the current disclosure, such as 8-Br-cAMP (Wang et al., 2011); M-CSF treatment (Li et al., 2016); exposure to reveresine (Li et al., 2016); and/or exposure to stem cell extracts (Xiong et al., 2014). Characterization of fibroblast dedifferentiation can be performed by assessment of extracellular markers, such as CXCR4, VEGFR-2, CD34, and/or CD133, as well as intracellular markers such as SOX-2, NANOG, and/or OCT-4.

In particular embodiments de-differentiated fibroblasts are utilized. Such fibroblasts are induced to de-differentiate, and the de-differentiated cells are manipulated to produce certain factor(s). In specific embodiments, induction of de-differentiation of the fibroblasts is performed by culture of the fibroblasts together with cytoplasm from a cell possessing a more undifferentiated phenotype, as compared to original fibroblasts. In some cases, de-differentiation of the fibroblasts is performed by culture of the fibroblasts with cells possessing a more undifferentiated phenotype. Cells possessing a more undifferentiated phenotype may be any kind of stem cell, for example, such as pluripotent stem cells,

In some embodiments of the disclosure, fibroblast cells that have been dedifferentiated may be utilized for the disclosed methods, wherein the cells express one or more markers selected from the group consisting of Telomerase, NANOG, Sox2, beta-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-alpha, HGF, c-MET, alpha-1-Antriptrypsin, Ceruloplasmin, AFP, PEPCK 1, BDNF, NT-4/5, TrkA, BMP2, BMP4, FGF2, FGF4, PDGF, PGF, TGFalpha, TGFbeta, VEGF, and a combination thereof.

In particular embodiments, culture of fibroblasts with undifferentiated cells (and/or cytoplasm from undifferentiated cells) is performed under conditions including the presence of one or more histone deacetylase inhibitors, such as a histone deacetylase inhibitor selected from a group consisting of: a) valproic acid; b) trichostatin A; c) phenylbutyrate; d) vorinostat; e) belinostat; f) LAQ824; g) panobinostat; h) entinostat; i) CI994; j) mocetinostat; k) sulforaphane; and l) a combination thereof. In specific cases exposure of the undifferentiated cells (and/or cytoplasm from undifferentiated cells) with one or more histone deacetylase inhibitors enhances the ability of the de-differentiated fibroblasts to cause regeneration of one or more discs of an individual.

In particular embodiments, culture of fibroblasts with undifferentiated cells (and/or cytoplasm from undifferentiated cells) is performed under conditions including the presence of one or more DNA methyltransferase inhibitors. The DNA methyltransferase inhibitor may be selected from the group consisting of: a) decitabine; b) 5-azacytidine; c) Zebularine; d) RG-108; e) procaine hydrochloride; f) Procainamide hydrochloride; g) Hydralazine hydrochloride; h) Epigallocatechin gallate; i) Chlorogenic acid; j) Caffeic acid; and h) a combination thereof. In specific cases exposure of the undifferentiated cells (and/or cytoplasm from undifferentiated cells) with one or more DNA methyltransferase inhibitors enhances the ability of the de-differentiated fibroblasts to cause regeneration of one or more discs of an individual.

In particular cases, media allowing for de-differentiated fibroblast proliferation comprises one or more factors known to be mitogenic for dedifferentiated fibroblasts, such as one or more factors selected from the group consisting of: a) FGF-1; b) FGF-2; c) FGF-5; d) EGF; e) CNTF; f) KGF-1; g) PDGF; h) platelet rich plasma; i) TGF-alpha; j) HGF-1; and k) a combination thereof. In specific cases exposure of the undifferentiated cells (and/or cytoplasm from undifferentiated cells) with one or more factors known to be mitogenic for dedifferentiated fibroblasts (for example, in culture) enhances the ability of the de-differentiated fibroblasts to cause regeneration of one or more discs of an individual.

In specific embodiments, fibroblasts subsequent to de-differentiation are cultured to obtained a conditioned media. In certain cases, the fibroblasts subsequent to de-differentiation are cultured that results in the production of exosomes from the de-differentiated cells, and exosomes are obtained from the conditioned media. In particular cases, the exosomes are collected from de-differentiated fibroblasts while the fibroblasts are in a proliferating state. Exosomes may be collected from de-differentiated fibroblasts while the de-differentiated fibroblasts are cultured in a media comprising no proliferative factors or largely reduced levels of proliferation-inducing growth factors. Exosomes may be collected from de-differentiated fibroblasts that have been cultured in media with certain levels of oxygen for a certain duration of time. For example, exosomes may be collected from de-differentiated fibroblasts that have been cultured in media with 2%-8%, 2%-7%, 2%-6%, 2%-5%, 2%-4%, 2%-3%, 3%-8%, 3%-7%, 3%-6%, 3%-5%, 3%-4%, 4%-8%, 4%-7%, 4%-6%, 4%-5%, 5%-8%, 5%-7%, 5%-6%, 6%-8%, 6%-7%, or 7%-8% oxygen, as examples. Exosomes may be collected from de-differentiated fibroblasts that have been cultured in media for a certain duration of time, and this duration may or may not include the above noted levels of oxygen. Exosomes may be collected from de-differentiated fibroblasts that have been cultured in media for at least 1, 2, 3, 4, 5, 6, 7, 8, 9, 10, 11, 12, 13, 14, or 15 days. The cells may be cultured for 1-15, 1-14, 1-13, 1-12, 1-11, 1-10, 1-9, 1-8, 1-7, 1-6, 1-5, 1-4, 1-3, 1-2, 2-15, 2-14, 2-13, 2-12, 2-11, 2-10, 2-9, 2-8, 2-7, 2-6, 2-5, 2-4, 2-3, 3-15, 3-14, 3-13, 3-12, 3-11, 3-10, 3-9, 3-8, 3-7, 3-6, 3-5, 3-4, 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, 13-15, 13-14, or 14-15 days, for example.

In one embodiment of the disclosure, fibroblasts to be used for immunomodulation are genetically engineered, for example to express: a) one or more autoantigens; and/or b) one or more immune modulatory proteins. The engineered cells are subsequently used for induction of immunological tolerance. The characteristics of the individual and disease dictate which genes are to be used for engineering of fibroblasts, in at least some cases. In these cases, the autoantigen may be transfected into the fibroblasts in polynucleotide form and the fibroblasts are either cultured to allow for immune modulation or transfected with genes allowing for immune modulation. Genes of particular interest for transfection to induce immune modulation include at least the following: Fas ligand, TGF-beta, IL-4, IL-10, HLA-G, indolamine 2,3 deoxygenase, galectin family members, Galectin 3, arginase, and/or IL-20 (de Jesus et al., 2016; Wang et al., 2011; Zhao et al., 2010; Min et al., 2001; Cancedda et al., 2001). Any of the genes described herein or active portions thereof may be cloned into mammalian expression constructs comprising promoter sequences enabling expression in fibroblast cells such as the CMV promoter (Artuc et al., Exp. Dermatol. 1995, 4:317-21). Examples of suitable constructs include, but are not limited to pcDNA3, pcDNA3.1 (+/−), pGL3, PzeoSV2 (+/−), pDisplay, pEF/myc/cyto, pCMV/myc/cyto (each of which is commercially available from vendors such as Invitrogen, for example), or the pSH expression vector that enables a regulated polynucleotide expression in human foreskin cells (Ventura and Villa, 1993, Biochem. Biophys. Commun. 192: 867-9). Examples of retroviral vector and packaging systems are those commercially available from Clontech, San Diego, Calif., USA, including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter. After completing plasmid transfection, fibroblasts are harvested by a means allowing for detachment from tissue culture plates, for example, by trypsinization and transferred to either a suitable vessel or container for proliferation. Approximately 3 days post-transfection, the cell media is changed to media suitable for proliferation and expansion of modified fibroblasts. One example is Neurobasal A (NBA) proliferation medium comprising Neurobasal-A (Invitrogen), 1% D-glucose (Sigma Aldrich), 1% Penicillin/Streptomycin/Glutamine (Invitrogen), 2% B27 supplement with Retinoic acid (Invitrogen), 0.2% EGF (Peprotech, USA), 0.08% FGF-2 (Peprotech), 0.2% Heparin (Sigma Aldrich, USA) and Valproic acid (Sigma Aldrich) to a concentration of 1 μM. The media is then subsequently changed, such as thrice weekly, and cells are re-plated regularly (for example, 2-8 times up to a maximum of weekly re-plating, becoming more regular as colonies began to develop) to remove non-reprogrammed cells until widespread colony formation is achieved. Various quality control means are known in the art for practitioners of the disclosure to perform clinical administration of the cells. Example criteria for qualification of the cells includes marker identification using means such as flow cytometry, viability, endotoxin content, as well as assessment for microbial and mycoplasma contamination.

In particular embodiments of the disclosure, fibroblasts are cultured ex vivo using means known in the art for preserving viability and proliferative ability of fibroblasts. The disclosure provides for the modification of known culture techniques to decrease recognition of fibroblasts by the recipient immune system. In one embodiment fibroblasts are cultured in conditions that lack xenogeneic components, such as fetal calf serum. Xenogeneic components are known to trigger immunological reactions, including elicitation of antibody and T cell reactions (Selvaggi et al., 1997; Mackensen et al., 2000; Kadri et al., 2007; Forni et al., 1976; Lauer et al., 1983). In many individuals, natural antibodies of the IgM isotype exist to fetal calf serum associated components (Irie et al., 1974), causing rejection, inflammation or anaphylaxis subsequent to administration of cells grown in the presence of fetal calf serum (Macy et al., 1989). In specific embodiments, the disclosure encompasses the substitution of fetal calf serum with human platelet rich plasma, platelet lysate, umbilical cord blood serum, autologous serum, and/or defined cytokine mixes as an additional feature to reduce the immunogenicity of fibroblasts. Means of culturing tissues in xenogeneic-free medium are known in the art for other cell types and are incorporated by reference (Riordan et al., 2015).

In one embodiment of the disclosure, fibroblasts are administered to a subject by any suitable route, including by injection (such as intramuscular injection), including in hypoxic areas. Suitable routes include intravenous, subcutaneous, intrathecal, oral, intrarectal, intrathecal, intra-omentral, intraventricular, intrahepatic, and intrarenal.

In certain embodiments, fibroblasts may be derived from tissues comprising skin, heart, blood vessels, bone marrow, skeletal muscle, liver, pancreas, brain, adipose tissue, foreskin, placental, and/or umbilical cord. In specific embodiments, the fibroblasts are placental, fetal, neonatal or adult or mixtures thereof.

The number of administrations of cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

Embodiments of the disclosure provide methods of reducing immunogenicity of particular types of fibroblasts. Fibroblasts may be derived from various tissues or organs, such as skin, heart, blood vessels, bone marrow, skeletal muscle, liver, pancreas, brain, and/or foreskin, which can be obtained by biopsy (where appropriate) or upon autopsy. In some aspects, the cells comprise fibroblasts, which can be from a fetal, neonatal, adult origin, or a combination thereof.

In some embodiments of the disclosure, genetic modification of fibroblasts is performed to cause reduction of immunogenicity of the fibroblasts. One method provides for genetic modification that includes cytoplasmic transfer with cells possessing reduced immunogenicity, such as immature dendritic cells. In another embodiment, gene editing is utilized to selectively excise inflammation-evoking genes, such as HLA or costimulatory molecules such as CD40, CD80, CD86, TNF-alpha, HMGB-1, IFN-gamma, IL-1 beta, IL-17, FAP, IL-18, IL-33, or a combination thereof.

In particular embodiments of the disclosure, one or more immunomodulatory agent(s) are expressed in universal donor fibroblasts via a recombinant expression vector operable in eukaryotic cells, and the expression of the immunomodulatory agent(s) may be regulated by a constitutive promoter or an inducible promoter or a tissue-specific promoter. In specific embodiments, the vector is a viral vector, such as a retrovirus, lentivirus, adenovirus, adeno-associated virus, or herpes simplex virus, or the vector is a non-viral vector, such as naked DNA or plasmid DNA or minicircle DNA. Non-viral vectors, such as plasmids or transposons, may be employed. Polynucleotides of particular interest for transfection into the fibroblasts include at least the following: Fas ligand, TGF-beta, IL-4, IL-10, HLA-G, indolamine 2,3 deoxygenase (IDO), galectin family members, Galectin 3, arginase, IL-20, HGF, PDGF-BB, EGF, IGF, GDF-5, GDF-11, Angiopoietin, FGF-1, FGF-2, FGF-5, FGF-15, or a combination thereof. In specific cases, recombinantly expressed angiogenic agent(s) may comprise FAS ligand, IL-2, IL-4, IL-10, IL-20, IL-35, HLA-G, 1-309, IDO, iNOS, CD200, Galectin 3, arginase, PGE-2, TGF-beta, CTLA-4, PD-L1, IFN-gamma, or combinations thereof.

In certain embodiments, a therapeutically effective amount of modified cells are co-administered with one or more immunomodulatory agents(s) to an individual. Exemplary immunomodulatory agents may comprise FAS ligand, IL-2R, IL-1 Ra, IL-2, IL-4, IL-8, IL-10, IL-20, IL-35, HLA-G, PD-L1, 1-309, IDO, iNOS, CD200, Galectin 3, sCR1, arginase, PGE-2, aspirin, atorvastatin, fluvastatin, lovastatin, pravastatin, rosuvastatin, simvastatin, pitavastatin, n-acetylcysteine, rapamycin, IVIG, naltrexone, TGF-beta, VEGF, PDGF, CTLA-4, anti-CD45RB antibody, hydroxychloroquine, leflunomide, auranofin, dicyanogold, sulfasalazine, methotrexate, glucocorticoids, etanercept, adalimumab, abatacept, anakinra, certolizumab, Etanercept-szzs, golimumab, infliximab, rituximab, tocilizumab, cyclosporine, IFN-gamma, everolimus, rapamycin, or combinations thereof.

In particular embodiments, methods are employed in that cells require no exogenous growth factors, except, in at least some cases, growth factors are available in the supplemental serum provided with the growth medium. Also provided herein are methods of deriving umbilical cells capable of expansion in the absence of particular growth factors. The methods are similar to the method above, however they may require that the particular growth factors (for which the cells have no requirement) be absent in the culture medium in which the cells are ultimately resuspended and grown. In this sense, the method is selective for those cells capable of division in the absence of the particular growth factors. Particular cells, in some embodiments, are capable of growth and expansion in chemically-defined growth media with no serum added. In such cases, the cells may require certain growth factors, which can be added to the medium to support and sustain the cells. Factors that may be added for growth on serum-free media may comprise one or more of FGF, EGF, IGF, and PDGF. In some embodiments, two, three or all four of the factors are added to serum-free or chemically defined media. In specific embodiments, leukemia inhibitory factor (LIF) is added to serum-free medium to support or improve growth of the cells.

### VII. Examples

### Example 1: Stimulation of Regeneration at a Disc Distal to a Disc Injected with Human Dermal Fibroblasts

Five patients with disc degenerative disease are administered intradiscally 10 million Human Dermal (CybroCell, for example) fibroblast cells. Administered cells are directed into the nucleus pulposus by means of fluoroscopy guided injection. After 6 months, regeneration is observed in the injected disc, as well as in discs proximal and distal to the area of administration.

### Example 2: Effective Treatment of Bilateral Osteoartheritis by Administration of Fibroblasts

Ten patients with osteoarthritis was administered 10 million CybroCell fibroblast cells intra-articularly in one knee. Three months after administration the injected knee demonstrates signs of cartilage regeneration. Additionally, the contralateral non-injected knee also generates signs of cartilage regeneration.

### REFERENCES

All publications mentioned in this specification are indicative of the level of those skilled in the art to which the invention pertains. All publications herein are incorporated by reference to the same extent as if each individual publication was specifically and individually indicated to be incorporated by reference in their entirety.

U.S. Pat. No. 4,353,888

U.S. Pat. No. 4,744,933

U.S. Pat. No. 4,749,620

U.S. Pat. No. 4,814,274

U.S. Pat. No. 5,084,350

U.S. Pat. No. 5,089,272

U.S. Pat. No. 5,578,442

U.S. Pat. No. 5,639,275

U.S. Pat. No. 5,676,943

U.S. Pat. No. 5,827,740

U.S. Pat. No. 6,326,198

U.S. Pat. No. 6,335,195

U.S. Pat. No. 6,338,942

U.S. Pat. No. 7,413,734

U.S. Pat. No. 7,510,873

U.S. Pat. No. 7,524,489

U.S. Pat. No. 7,560,276

U.S. Pub. 2013/0149286

European Patent Publication No. 301,777

1. Martin, I., et al., A relativity concept in mesenchymal stromal cell manufacturing. Cytotherapy, 2016. 18(5): p. 613-20.

2. Uder, C., et al., Mammalian MSC from selected species: Features and applications. Cytometry A, 2017.

3. Lechanteur, C., et al., Clinical-scale expansion of mesenchymal stromal cells: a large banking experience. J Transl Med, 2016. 14(1): p. 145.

4. Yi, T., et al., Manufacture of Clinical-Grade Human Clonal Mesenchymal Stem Cell Products from Single Colony Forming Unit-Derived Colonies Based on the Subfractionation Culturing Method. Tissue Eng Part C Methods, 2015. 21(12): p. 1251-62.

5. Hanley, P. J., Therapeutic mesenchymal stromal cells: where we are headed. Methods Mol Biol, 2015. 1283: p. 1-11.

6. Nold, P., et al., Good manufacturing practice-compliant animal-free expansion of human bone marrow derived mesenchymal stroma cells in a closed hollow-fiber-based bioreactor. Biochem Biophys Res Commun, 2013. 430(1): p. 325-30.

7. Gastens, M. H., et al., Good manufacturing practice-compliant expansion of marrow-derived stem and progenitor cells for cell therapy. Cell Transplant, 2007. 16(7): p. 685-96.

8. Nicoletti, G. F., et al., Methods and procedures in adipose stem cells: state of the art and perspective for translation medicine. J Cell Physiol, 2015. 230(3): p. 489-95.

9. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.

10. Gubar, O. S., et al., Postnatal extra-embryonic tissues as a source of multiple cell types for regenerative medicine applications. Exp Oncol, 2017. 39(3): p. 186-190.

11. Lim, R., et al., A Pilot Study Evaluating the Safety of Intravenously Administered Human Amnion Epithelial Cells for the Treatment of Hepatic Fibrosis. Front Pharmacol, 2017. 8: p. 549.

12. Zlatska, A. V., et al., Endometrial stromal cells: isolation, expansion, morphological and functional properties. Exp Oncol, 2017. 39(3): p. 197-202.

13. Lan, X., et al., Stromal Cell-Derived Factor-1 Mediates Cardiac Allograft Tolerance Induced by Human Endometrial Regenerative Cell-Based Therapy. Stem Cells Trans1 Med, 2017.

14. Xu, X., et al., Prolongation of Cardiac Allograft Survival by Endometrial Regenerative Cells: Focusing on B-Cell Responses. Stem Cells Transl Med, 2017. 6(3): p. 778-787.

15. Rodrigues, M. C., et al., Menstrual Blood-Derived Stem Cells: In Vitro and In Vivo Characterization of Functional Effects. Adv Exp Med Biol, 2016. 951: p. 111-121.

16. James, J. L., et al., Isolation and characterisation of a novel trophoblast side-population from first trimester placentae. Reproduction, 2015. 150(5): p. 449-62.

17. Wang, Z., et al., Transplantation of human villous trophoblasts preserves cardiac function in mice with acute myocardial infarction. J Cell Mol Med, 2017. 21(10): p. 2432-2440.

18. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.

19. Ducret, M., et al., A standardized procedure to obtain mesenchymal stem/stromal cells from minimally manipulated dental pulp and Wharton's jelly samples. Bull Group Int Rech Sci Stomatol Odontol, 2016. 53(1): p. e37.

20. Van Pham, P., et al., Isolation and proliferation of umbilical cord tissue derived mesenchymal stem cells for clinical applications. Cell Tissue Bank, 2016. 17(2): p. 289-302.

21. Friedman, R., et al., Umbilical cord mesenchymal stem cells: adjuvants for human cell transplantation. Biol Blood Marrow Transplant, 2007. 13(12): p. 1477-86.

22. Emnett, R. J., et al., Evaluation of Tissue Homogenization to Support the Generation of GMP-Compliant Mesenchymal Stromal Cells from the Umbilical Cord. Stem Cells Int, 2016. 2016: p. 3274054.

23. Choi, Y. S., et al., Different characteristics of mesenchymal stem cells isolated from different layers of full term placenta. PLoS One, 2017. 12(2): p. e0172642.

24. Koike, C., et al., Characterization of amniotic stem cells. Cell Reprogram, 2014. 16(4): p. 298-305.

25. Kim, S. W., et al., Amniotic mesenchymal stem cells with robust chemotactic properties are effective in the treatment of a myocardial infarction model. Int J Cardiol, 2013. 168(2): p. 1062-9.

26. Walther, G., J. Gekas, and O. F. Bertrand, Amniotic stem cells for cellular cardiomyoplasty: promises and premises. Catheter Cardiovasc Interv, 2009. 73(7): p. 917-24.

27. Ullah, M., et al., iPS-derived MSCs from an expandable bank to deliver a prodrug-converting enzyme that limits growth and metastases of human breast cancers. Cell Death Discov, 2017. 3: p. 16064.

28. Moslem, M., et al., Kindlin-2 Modulates the Survival, Differentiation, and Migration of Induced Pluripotent Cell-Derived Mesenchymal Stromal Cells. Stem Cells Int, 2017. 2017: p. 7316354.

29. Luzzani, C. D. and S. G. Miriuka, Pluripotent Stem Cells as a Robust Source of Mesenchymal Stem Cells. Stem Cell Rev, 2017. 13(1): p. 68-78.

30. Lo Cicero, A., et al., A High Throughput Phenotypic Screening reveals compounds that counteract premature osteogenic differentiation of HGPS iPS-derived mesenchymal stem cells. Sci Rep, 2016. 6: p. 34798.

31. Sheyn, D., et al., Human Induced Pluripotent Stem Cells Differentiate Into Functional Mesenchymal Stem Cells and Repair Bone Defects. Stem Cells Transl Med, 2016. 5(11): p. 1447-1460.

32. Shi, S., et al., Bone formation by human postnatal bone marrow stromal stem cells is enhanced by telomerase expression. Nat Biotechnol, 2002. 20(6): p. 587-91.

33. Grau-Monge, C., et al., Marrow-isolated adult multilineage inducible cells embedded within a biologically-inspired construct promote recovery in a mouse model of peripheral vascular disease. Biomed Mater, 2017. 12(1): p. 015024.

34. Rahnemai-Azar, A., et al., Human marrow-isolated adult multilineage-inducible (MIAMI) cells protect against peripheral vascular ischemia in a mouse model. Cytotherapy, 2011. 13(2): p. 179-92.

35. Soeder, Y., et al., First-in-Human Case Study: Multipotent Adult Progenitor Cells for Immunomodulation After Liver Transplantation. Stem Cells Transl Med, 2015. 4(8): p. 899-904.

36. Boozer, S., et al., Global Characterization and Genomic Stability of Human MultiStem, A Multipotent Adult Progenitor Cell. J Stem Cells, 2009. 4(1): p. 17-28.

37. Maziarz, R. T., et al., Single and multiple dose MultiStem (multipotent adult progenitor cell) therapy prophylaxis of acute graft-versus-host disease in myeloablative allogeneic hematopoietic cell transplantation: a phase 1 trial. Biol Blood Marrow Transplant, 2015. 21(4): p. 720-8.

38. Plessers, J., et al., Clinical-Grade Human Multipotent Adult Progenitor Cells Block CD8+Cytotoxic T Lymphocytes. Stem Cells Transl Med, 2016. 5(12): p. 1607-1619.

39. Kebriaei, P., et al., Adult human mesenchymal stem cells added to corticosteroid therapy for the treatment of acute graft-versus-host disease. Biol Blood Marrow Transplant, 2009. 15(7): p. 804-11.

40. Allison, M., Genzyme backs Osiris, despite Prochymal flop. Nat Biotechnol, 2009. 27(11): p. 966-7.

41. Hare, J. M., et al., A randomized, double-blind, placebo-controlled, dose-escalation study of intravenous adult human mesenchymal stem cells (prochymal) after acute myocardial infarction. J Am Coll Cardiol, 2009. 54(24): p. 2277-86.

42. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.

43. Patel, A. N. and J. Genovese, Potential clinical applications of adult human mesenchymal stem cell (Prochymal(R)) therapy. Stem Cells Cloning, 2011. 4: p. 61-72.

44. Mannon, P. J., Remestemcel-L: human mesenchymal stem cells as an emerging therapy for Crohn's disease. Expert Opin Biol Ther, 2011. 11(9): p. 1249-56.

45. Wang, Y., et al., Safety, tolerability, clinical, and joint structural outcomes of a single intra-articular injection of allogeneic mesenchymal precursor cells in patients following anterior cruciate ligament reconstruction: a controlled double-blind randomised trial. Arthritis Res Ther, 2017. 19(1): p. 180.

46. Kolar, M. K., et al., The neurotrophic effects of different human dental mesenchymal stem cells. Sci Rep, 2017. 7(1): p. 12605.

47. Prather, W. R., A. Toren, and M. Meiron, Placental-derived and expanded mesenchymal stromal cells (PLX-I) to enhance the engraftment of hematopoietic stem cells derived from umbilical cord blood. Expert Opin Biol Ther, 2008. 8(8): p. 1241-50.

48. Patel, A. N., et al., Ixmyelocel-T for patients with ischaemic heart failure: a prospective randomised double-blind trial. Lancet, 2016. 387(10036): p. 2412-21.

49. Perets, N., et al., Long term beneficial effect of neurotrophic factors-secreting mesenchymal stem cells transplantation in the BTBR mouse model of autism. Behav Brain Res, 2017. 331: p. 254-260.

50. Gupta, P. K., et al., Administration of Adult Human Bone Marrow-Derived, Cultured, Pooled, Allogeneic Mesenchymal Stromal Cells in Critical Limb Ischemia Due to Buerger's Disease: Phase II Study Report Suggests Clinical Efficacy. Stem Cells Transl Med, 2017. 6(3): p. 689-699.

51. Thej, C., et al., Development of a surrogate potency assay to determine the angiogenic activity of Stempeucel(R), a pooled, ex-vivo expanded, allogeneic human bone marrow mesenchymal stromal cell product. Stem Cell Res Ther, 2017. 8(1): p. 47.

52. Zhu, M., et al., Manual isolation of adipose-derived stem cells from human lipoaspirates. J Vis Exp, 2013(79): p. e50585.

53. Van Pham, P., et al., Isolation and proliferation of umbilical cord tissue derived mesenchymal stem cells for clinical applications. Cell Tissue Bank, 2015.

54. Fazzina, R., et al., A new standardized clinical-grade protocol for banking human umbilical cord tissue cells. Transfusion, 2015. 55(12): p. 2864-73.

55. Bieback, K., Platelet lysate as replacement for fetal bovine serum in mesenchymal stromal cell cultures. Transfus Med Hemother, 2013. 40(5): p. 326-35.

56. Stanko, P., et al., Comparison of human mesenchymal stem cells derived from dental pulp, bone marrow, adipose tissue, and umbilical cord tissue by gene expression.Biomed Pap Med Fac Univ Palacky Olomouc Czech Repub, 2014. 158(3): p. 373-7.

57. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.

58. Can, A., F. T. Celikkan, and O. Cinar, Umbilical cord mesenchymal stromal cell transplantations: A systemic analysis of clinical trials. Cytotherapy, 2017.

59. Bilal, M., A. Haseeb, and M. A. Sher Khan, Intracoronary infusion of Wharton's jelly-derived mesenchymal stem cells: a novel treatment in patients of acute myocardial infarction. J Pak Med Assoc, 2015. 65(12): p. 1369.

60. Gao, L. R., et al., Intracoronary infusion of Wharton's jelly-derived mesenchymal stem cells in acute myocardial infarction: double-blind, randomized controlled trial. BMC Med, 2015. 13: p. 162.

61. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.

62. Liu, X., et al., A preliminary evaluation of efficacy and safety of Wharton's jelly mesenchymal stem cell transplantation in patients with type 2 diabetes mellitus. Stem Cell Res Ther, 2014. 5(2): p. 57.

63. Wu, K. H., et al., Human application of ex vivo expanded umbilical cord-derived mesenchymal stem cells: enhance hematopoiesis after cord blood transplantation. Cell Transplant, 2013. 22(11): p. 2041-51.

64. Kim, D. W., et al., Wharton's jelly-derived mesenchymal stem cells: phenotypic characterization and optimizing their therapeutic potential for clinical applications. Int J Mol Sci, 2013. 14(6): p. 11692-712.

65. Batsali, A. K., et al., Mesenchymal stem cells derived from Wharton's Jelly of the umbilical cord: biological properties and emerging clinical applications. Curr Stem Cell Res Ther, 2013. 8(2): p. 144-55.

66. Hu, J., et al., Long term effects of the implantation of Wharton's jelly-derived mesenchymal stem cells from the umbilical cord for newly-onset type 1 diabetes mellitus. Endocr J, 2013. 60(3): p. 347-57.

67. de Soure, A. M., et al., Scalable microcarrier-based manufacturing of mesenchymal stem/stromal cells. J Biotechnol, 2016. 236: p. 88-109.

68. Fernandes-Platzgummer, A., et al., Clinical-Grade Manufacturing of Therapeutic Human Mesenchymal Stem/Stromal Cells in Microcarrier-Based Culture Systems. Methods Mol Biol, 2016. 1416: p. 375-88.

69. Mizukami, A., et al., Stirred tank bioreactor culture combined with serum/xenogeneic-free culture medium enables an efficient expansion of umbilical cord-derived mesenchymal stem/stromal cells. Biotechnol J, 2016. 11(8): p. 1048-59.

70. Smith, J. R., et al., Standardizing Umbilical Cord Mesenchymal Stromal Cells for Translation to Clinical Use: Selection of GMP-Compliant Medium and a Simplified Isolation Method. Stem Cells Int, 2016. 2016: p. 6810980.

71. Carmelo, J. G., et al., Scalable ex vivo expansion of human mesenchymal stem/stromal cells in microcarrier-based stirred culture systems. Methods Mol Biol, 2015. 1283: p. 147-59.

72. Fekete, N., et al., GMP-compliant isolation and large-scale expansion of bone marrow-derived MSC. PLoS One, 2012. 7(8): p. e43255.

73. Lange, C., et al., Accelerated and safe expansion of human mesenchymal stromal cells in animal serum free medium for transplantation and regenerative medicine. J Cell Physiol, 2007. 213(1): p. 18-26.

74. Tanthaisong, P., et al., Enhanced Chondrogenic Differentiation of Human Umbilical Cord Wharton's Jelly Derived Mesenchymal Stem Cells by GSK-3 Inhibitors. PLoS One, 2017. 12(1): p. e0168059.

75. Linares, G. R., et al., Preconditioning mesenchymal stem cells with the mood stabilizers lithium and valproic acid enhances therapeutic efficacy in a mouse model of Huntington's disease. Exp Neurol, 2016. 281: p. 81-92.

76. Ferensztajn-Rochowiak, E., et al., The effect of long-term lithium treatment of bipolar disorder on stem cells circulating in peripheral blood. World J Biol Psychiatry, 2017. 18(1): p. 54-62.

77. Ferensztajn-Rochowiak, E. and J. K. Rybakowski, The effect of lithium on hematopoietic, mesenchymal and neural stem cells. Pharmacol Rep, 2016. 68(2): p. 224-30.

78. Dong, B. T., et al., Lithium enhanced cell proliferation and differentiation of mesenchymal stem cells to neural cells in rat spinal cord. Int J Clin Exp Pathol, 2015. 8(3): p. 2473-83.

79. Zhu, Z., et al., Lithium stimulates human bone marrow derived mesenchymal stem cell proliferation through GSK-3beta-dependent beta-catenin/Wnt pathway activation. FEBS J, 2014. 281(23): p. 5371-89.

80. Tsoyi, K., et al., Carbon Monoxide Improves Efficacy of Mesenchymal Stromal Cells During Sepsis by Production of Specialized Proresolving Lipid Mediators. Crit Care Med, 2016. 44(12): p. e1236-e1245.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of stimulating regeneration in a first tissue site in an individual, comprising the step of administering at least one regenerative composition comprising fibroblasts and/or dedifferentiated fibroblast cells and optionally stem cells to a second tissue site, wherein the second tissue site comprises the same tissue type as the first tissue site in the individual.

2. The method of claim 1, wherein administering the composition comprises systemic injection, local injection, systemic delivery, and/or local delivery.

3. The method of claim 1 or 2, wherein administering the composition comprises at least one administration or 2, 3, 4, 5, 6, 7, 8, 9, 10 or more administrations.

4. The method of any one of claims 1-3, wherein the first tissue has partial or full loss of functionality.

5. The method of claim 4, wherein loss of functionality comprises cell death in the tissue, tissue necrosis, atrophy, fibrosis, inflammation, fat deposition, generation of degenerative molecules, loss of elasticity, neurodegeneration, autoimmunity, complement activation, cartilage loss, ligament tear(s), muscle tear(s), loss of connective tissue, neoplasm(s), or a combination thereof.

6. The method of any one of claims 1-5, wherein the tissue is comprised of muscle tissue, connective tissue, epithelial tissue, endothelial tissue, nervous tissue, fat tissue, skin tissue, lung tissue, liver tissue, bladder tissue, kidney tissue, heart tissue, stomach tissue, intestinal tissue, spinal tissue, eye tissue, fibrous tissue, omentum, lymphatic tissue, bone marrow, or a combination thereof.

7. The method of any one of claims 1-6, wherein said tissue is comprised of one or more cells selected from the group consisting of endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, interstitial kidney cells, and a combination thereof.

8. The method of any one of claims 1-7, wherein the regenerative composition comprises at least one growth factor.

9. The method of claim 8, wherein at least one growth factor is selected from a group consisting of AM, Ang, BMP, BDNF, EGF, Epo, FGF, GNDF, G-CSF, GM-CSF, GDF-9, HGF, HDGF, IGF, migration-stimulating factor, GDF-8, GDF-11, GDF-15, MGF, NGF, P1GF, PDGF, Tpo, TGF-alpha, TGF-beta, TNF-alpha, VEGF, a Wnt protein, an interleukin, a soluble receptor for IL-1alpha, IL-1beta, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12, 35 kDa alpha subunit, IL-12, 40 kDa beta subunit, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-24, IL-25, IL-26, IL-27B, IL-27-p28, IL-28A, IL-28B, IL-29, IL-30, IL-31, IL-32, IL-33, IL-34, IL-35, IL-36alpha, IL-36beta, IL-36gamma, an interferon (IFN), a soluble receptor for IFN-alpha, IFN-beta, IFN-gamma, IFN-lamdal, IFN-lamda2, IFN-lamda3, IFN-K, IFN-epsilon, IFN-kappa, IFN-tau, IFN-delta, IFN-zeta, IFN-omega, IFN-v, insulin, proinsulin, a receptor for insulin, leptin (LEP), and a combination thereof.

10. The method of any one of claims 1-9, wherein the regenerative composition comprises platelet rich plasma.

11. The method of claim 10, wherein the platelet rich plasma comprises platelet lysate.

12. The method of claim 10, wherein the platelet rich plasma is derived from peripheral blood.

13. The method of claim 10, wherein the platelet rich plasma is derived from cord blood.

14. The method of any one of claims 1-13, wherein the regenerative composition comprises one or more exosome(s) derived from at least one regenerative cell.

15. The method of claim 14, wherein the regenerative cell is a stem cell and/or a fibroblast.

16. The method of claim 15, wherein the fibroblast is derived from tissue sources selected from the group consisting of foreskin, adipose tissue, placenta, ear lobe, omentum, wharton's jelly, and a combination thereof.

17. The method of any one of claims 14-16, wherein said exosomes possess a size of between 2 nm and 200 nm.

18. The method of any one of claims 14-17, wherein said exosomes possess a size between 30 and 150 nm

19. The method of any one of claims 14-18, wherein the exosomes comprise at least one lipid selected from the group consisting of phospholipids, phosphatidyl serine, phosphatidyl inositol, phosphatidyl choline, sphingomyelin, ceramides, glycolipid, cerebroside, steroids, cholesterol, and a combination thereof.

20. The method of any one of claims 14-19, wherein said exosomes comprise at least one lipid raft.

21. The method of any one of claims 14-20, wherein said exosomes comprise one or more antigenic markers on a surface of said exosomes selected from the group consisting of CD9, CD63, CD81, ANXA2, ENO1, HSP9OAA1, EEF1A1, YWHAE, SDCBP, PDCD6IP, ALB, YWHAZ, EEF2, ACTG1, LDHA, HSP90AB1, ALDOA, MSN, ANXA5, PGK1, CFL1, and a combination thereof.

22. The method of any one of claims 1-21, wherein the regenerative composition comprises one or more fibroblasts.

23. The method of claim 22, wherein the fibroblast is derived from tissue sources selected from the group consisting of foreskin, adipose tissue, placenta, ear lobe, adipose tissue, omentum, wharton's jelly, and a combination thereof.

24. The method of claim 22, or 23, wherein said fibroblasts express at least one marker selected from the group consisting of NANOG, OCT-4, SSEA-4, stem cell factor receptor, and a combination thereof.

25. The method of any one of claims 1-24, wherein the regenerative composition comprises one or more stem cells.

26. The method of 25, wherein the stem cells comprise pluripotent stem cells.

27. The method of claim 26, wherein the pluripotent stem cells are selected from the group consisting of embryonic stem cells, parthenogenic derived stem cells, inducible pluripotent stem cells, somatic cell nuclear transfer derived stem cells, cytoplasmic transfer derived stem cells, stimulus-triggered acquisition of pluripotency, and a combination thereof.

28. The method of 25, wherein the stem cells comprise hematopoietic stem cells.

29. The method of claim 28, wherein the hematopoietic stem cells are capable of multi-lineage reconstitution in an immunodeficient host.

30. The method of claim 28, wherein the hematopoietic stem cells express at least one of the proteins selected from the group consisting of c-kit, Sca-1, CD34, CD133, and a combination thereof.

31. The method of any one of claims 28-30, wherein the hematopoietic stem cell expresses the Sca-1 protein.

32. The method of any one of claims 28-31, wherein said hematopoietic stem cells express CD34.

33. The method of any one of claims 28-32, wherein said hematopoietic stem cells express CD133.

34. The method of claim 28, wherein said hematopoietic stem cells lack expression of one or more lineage markers.

35. The method of claim 28, wherein said hematopoietic stem cells lack expression of CD38, CD14, CD16, CD56, or a combination thereof.

36. The method of claim 28, wherein the hematopoietic stem cell is positive for expression of c-kit, positive for expression of Sca-1, and/or substantially lacks expression of lineage markers.

37. The method of claim 28, wherein the hematopoietic stem cell is derived from sources selected from the group consisting of peripheral blood, mobilized peripheral blood, bone marrow, cord blood, adipose stromal vascular fraction, derived from progenitor cells, and a combination thereof.

38. The method of claim 32, wherein said hematopoietic progenitor cell is a pluripotent stem cell.

39. The method of claim 25, wherein the stem cells comprises mesenchymal stem cells.

40. The method of claim 39, wherein the mesenchymal stem cells are plastic adherent.

41. The method of claim 39 or 40, wherein the mesenchymal stem cells express a marker selected from the group consisting of CD73, CD90, CD105, and a combination thereof.

42. The method of claim 39, 40, or 41, wherein the mesenchymal stem cells lack expression of a marker selected from the group consisting of CD14, CD45, CD34, and a combination thereof.

43. The method of claim 39, wherein the mesenchymal stem cells are derived from tissues selected from the group consisting of bone marrow, peripheral blood, adipose tissue, mobilized peripheral blood, umbilical cord blood, Wharton's jelly, umbilical cord tissue, skeletal muscle tissue, subepithelial umbilical cord, endometrial tissue, menstrual blood, fallopian tube tissue, and a combination thereof.

44. The method of claim 43, wherein the mesenchymal stem cells derived from umbilical cord tissue express markers selected from the group consisting of oxidized low density lipoprotein receptor 1, chemokine receptor ligand 3, granulocyte chemotactic protein, and a combination thereof.

45. The method of claim 43 or 44, wherein the mesenchymal stem cells from umbilical cord tissue do not express markers selected from the group consisting of CD117, CD31, CD34, CD45, and a combination thereof.

46. The method of any one of claims 43-45, wherein the mesenchymal stem cells from umbilical cord tissue express, relative to a human fibroblast, increased levels of interleukin 8 and/or reticulon 1.

47. The method of any one of claims 43-46, wherein the mesenchymal stem cells from umbilical cord tissue express markers selected from the group consisting of CD10, CD13, CD44, CD73, CD90, and a combination thereof.

48. The method of any one of claims 43-47, wherein the umbilical cord tissue-derived cell secretes factors selected from the group consisting of MCP-1, MIP1beta, IL-6, IL-8, GCP-2, HGF, KGF, FGF, HB-EGF, BDNF, TPO, RANTES, TIMP1, and a combination thereof.

49. The method of any one of claims 43-48, wherein the umbilical cord tissue-derived cells express markers selected from the group consisting of TRA1-60, TRA1-81, SSEA3, SSEA4, NANOG, and a combination thereof.

50. The method of any one of claims 43-49, wherein the umbilical cord tissue-derived mesenchymal stem cells are isolated umbilical cord tissue cells isolated from umbilical cord tissue substantially free of blood that is capable of self-renewal and expansion in culture.

51. The method of any one of claims 43-50, wherein the umbilical cord tissue-derived cells are positive for alkaline phosphatase staining.

52. The method of claim 43, wherein the cord tissue-derived mesenchymal stem cells can undergo at least 20 doublings in culture.

53. The method of claim 49, wherein the cord tissue-derived mesenchymal stem cells maintain a normal karyotype upon passaging.

54. The method of any one of claims 43-53, wherein the umbilical cord tissue-derived mesenchymal stem cells express a marker selected from the group consisting of CD10, CD13, CD44, CD73, CD90, PDGFr-alpha, PD-L2, HLA-A,B,C, and a combination thereof.

55. The method of any one of claims 43-51, wherein the cord tissue-derived mesenchymal stem cells do not express one or more markers selected from the group consisting of CD31, CD34, CD45, CD80, CD86, CD117, CD141, CD178, B7-H2, HLA-G, HLA-DR,DP,DQ, and a combination thereof.

56. The method of claim 43, wherein the bone marrow-derived mesenchymal stem cells express markers selected from the group consisting of LFA-3, ICAM-1, PECAM-1, P-selectin, L-selectin, CD49b/CD29, CD49c/CD29, CD49d/CD29, CD29, CD18, CD61, 6-19, thrombomodulin, telomerase, CD10, CD13, CD34, CD56, CD117, integrin beta, and a combination thereof.

57. The method of any one of claim 43 or 56, wherein the bone marrow mesenchymal stem cells do not express CD10.

58. The method of any one of claim 43, 56, or 57, wherein the bone marrow mesenchymal stem cells do not express at least one of CD2, CDS, CD14, CD19, CD33, CD45, and/or DRII.

59. The method of any one of claim 43, 56, 57, or 58, wherein the bone marrow mesenchymal stem cells express at least one of CD13,CD34, CD56, CD90, CD117 and/or nestin.

60. The method of any one of claims 43-59, wherein the bone marrow-derived mesenchymal stem cells comprise mesenchymal stem cell progenitor cells.

61. The method of claim 60, wherein the mesenchymal progenitor cells comprise a population of bone marrow mesenchymal stem cells enriched for cells expressing STRO-1.

62. The method of any one of claim 60 or 61, wherein the mesenchymal progenitor cells express both STRO-1 and VCAM-1.

63. The method of any one of claims 60-62, wherein the STRO-1 expressing cells are negative for at least one marker selected from the group consisting of CBFA-1, collagen type II, PPAR.gamma2, osteopontin, osteocalcin, parathyroid hormone receptor, leptin, H-ALBP, aggrecan, Ki67, glycophorin A, and a combination thereof.

64. The method of claim 60, wherein the bone marrow mesenchymal stem cells lack expression of at least one of CD14, CD34, and/or CD45.

65. The method of any one of claims 61-64, wherein the STRO-1 expressing cells are positive for a marker selected from the group consisting of VCAM-1, TKY-1, CD146, STRO-2, and the combination thereof.

66. The method of claim 43, wherein the skeletal muscle stem cells express markers selected from the group consisting of CD13, CD34, CD56, CD117, and a combination thereof.

67. The method of any one of claim 43 or 66, wherein the skeletal muscle mesenchymal stem cells do not express CD10.

68. The method of any one of claim 43, 66, or 67, wherein the skeletal muscle mesenchymal stem cells do not express at least one of CD2, CDS, CD14, CD19, CD33, CD45, and/or DRII.

69. The method of claim 43, wherein the subepithelial umbilical cord-derived mesenchymal stem cells possess markers selected from the group consisting of CD29, CD73, CD90, CD166, SSEA4, CD9, CD44, CD146, CD105, and a combination thereof.

70. The method of any one of claim 43 or 69, wherein the subepithelial umbilical cord derived mesenchymal stem cells do not express markers selected from the group consisting of CD45, CD34, CD14, CD79, CD106,CD86, CD80, CD19, CD117, Stro-1, HLA-DR, and a combination thereof.

71. The method of any one of claim 43, 69, or 70, wherein the subepithelial umbilical cord derived mesenchymal stem cells express at least one of CD29, CD73, CD90, CD166, SSEA4, CD9, CD44, CD146, and/or CD105.

72. The method of any one of claim 43, or 69-71, wherein said subepithelial umbilical cord derived mesenchymal stem cells do not express at least one of CD45, CD34, CD14, CD79, CD106, CD86, CD80, CD19, CD117, Stro-1, and/or HLA-DR.

73. The method of any one of claim 43, or 69-72, wherein said subepithelial umbilical cord derived mesenchymal stem cells are positive for SOX2.

74. The method of any one of claim 43, or 69-73, wherein said subepithelial umbilical cord derived mesenchymal stem cells are positive for OCT4.

75. The method of any one of claim 43, or 69-74, wherein said subepithelial umbilical cord derived mesenchymal stem cells are positive for OCT4 and SOX2.

76. The method of any one of claims 1-75, wherein the regenerative composition comprises one or more fibroblast derived apoptotic vesicles.

77. The method of any one of claims 1-76, wherein the regenerative composition comprises fibroblast-derived miRNAs.

78. The method of claim 77, wherein said fibroblast derived miRNAs are comprised in exosomes.

79. The method of claim 77, wherein said fibroblast derived miRNAs are comprised in apoptotic bodies.

80. The method of claim 77, wherein said fibroblast derived miRNAs are circulating in plasma.

81. The method of any one of claims 1-80, wherein enhancement of one or more distant regenerative effects is accomplished by systemic administration of one or more epigenetic acting compositions.

82. The method of claim 81, wherein systemic administration occurs at the site of administration of the regenerative composition or at a different site.

83. The method of claim 81 or 82, wherein said epigenetic-acting composition comprises one or more histone deacetylase inhibitors.

84. The method of any one of claims 81=83, wherein said epigenetic-acting composition comprises one or more DNA methyltransferase inhibitors.

85. The method of claim 4, wherein the first tissue is degenerated.
